(19) United States
(12) Patent Application Publication
(10) Pub. No.: US 2025/0230162 A1
(43) Pub. Date: Jul. 17, 2025

(54) METHODS AND INTERMEDIATES FOR PREPARING THERAPEUTIC COMPOUNDS

(71) Applicant: Gilead Sciences, Inc., Foster City, CA (US)

(72) Inventors: Eric J. Alexy, Belmont, CA (US); Angie R. Angeles, San Diego, CA (US); Dustin A. Bringley, San Carlos, CA (US); Daniel R. Holycross, Daly City, CA (US); Erik Rueden, San Mateo, CA (US); Justin Y. Su, Foster City, CA (US)

(21) Appl. No.: 18/987,447

(22) Filed: Dec. 19, 2024

Related U.S. Application Data
(60) Provisional application No. 63/613,780, filed on Dec. 22, 2023.

(51) Int. Cl. C07D 471/18

(52) U.S. Cl. CPC C07D 471/18

(57) ABSTRACT

The present disclosure relates to methods and intermediates useful for preparing a compound of Formula I:

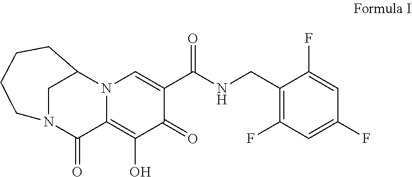 [SMILES: O=C(NCc1c(F)cc(F)cc1F)c1cn2c(c(O)c1=O)C(=O)N1CCCCC2C1]

or a salt thereof.

### CROSS REFERENCE TO RELATED APPLICATIONS

This application claims the benefit of U.S. Provisional Application No. 63/613,780, filed on Dec. 22, 2023, the entire contents of which is hereby incorporated by reference in its entirety.

### FIELD

The present disclosure relates to methods and intermediates for the synthesis of novel bridged tricyclic carbamoylpyridone compounds for use in the treatment of human immunodeficiency virus (HIV) infection.

### BACKGROUND

The present disclosure relates generally to the field of organic synthetic methodology for the preparation of antiviral compounds and their synthetic intermediates.

Human immunodeficiency virus infection and related diseases are a major public health problem worldwide. Human immunodeficiency virus encodes three enzymes which are required for viral replication: reverse transcriptase, protease, and integrase. Although drugs targeting reverse transcriptase and protease are in wide use and have shown effectiveness, particularly when employed in combination, toxicity and development of resistant strains may limit their usefulness (Palella, et al. N. Engl. J Med. (1998) 338:853-860; Richman, D. D. Nature (2001) 410:995-1001). Accordingly, there is a need for new agents that inhibit the replication of HIV.

U.S. Pat. No. 11,084,832 discloses novel compounds useful for treating an infection caused by the HIV virus. One specific compound identified therein is a compound of Formula I:

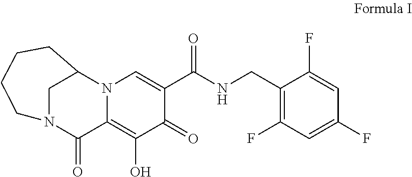 [SMILES: O=C(NCc1c(F)cc(F)cc1F)c1cn2c(c(O)c1=O)C(=O)N1CCCCC2C1]

There is currently a need for synthetic methods and intermediates that can be used to prepare the compound of Formula I and salts thereof.

### SUMMARY

The present disclosure is directed to a novel synthetic process for preparing a compound of Formula I:

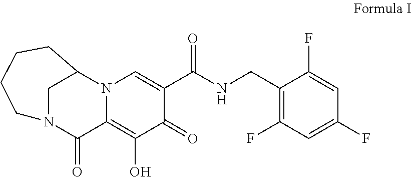 [SMILES: O=C(NCc1c(F)cc(F)cc1F)c1cn2c(c(O)c1=O)C(=O)N1CCCCC2C1]

or a salt thereof. The compound of Formula I may also be named or identified as 12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide.

The present disclosure is also directed to particular individual steps of this process and intermediates used in this process.

In some embodiments, disclosed herein is a process for preparing a compound of Formula I:

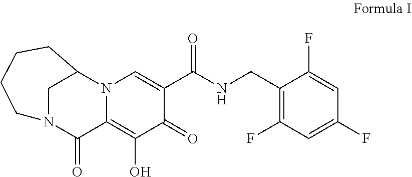 [SMILES: O=C(NCc1c(F)cc(F)cc1F)c1cn2c(c(O)c1=O)C(=O)N1CCCCC2C1]

comprising treating a compound of Formula A:

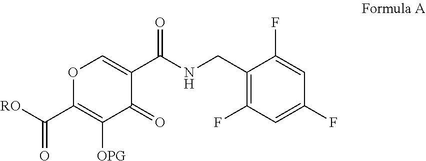

with a compound of Formula B:

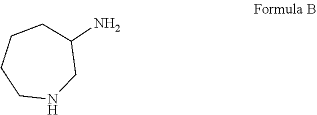

or a salt thereof, wherein PG is a hydroxyl protecting group and R is C₁₋₆ alkyl, C₆₋₁₀ aryl-C₁₋₄ alkylene, or C₆₋₁₀ aryl.

Additional embodiments of the disclosure, including additional synthetic intermediates and methods for preparing such intermediates, are provided herein.

### DETAILED DESCRIPTION

The description below is made with the understanding that the present disclosure is to be considered as an exemplification of the claimed subject matter, and is not intended to limit the appended claims to the specific embodiments illustrated. The headings used throughout this disclosure are provided for convenience and are not to be construed to limit the claims in any way. Embodiments illustrated under any heading may be combined with embodiments illustrated under any other heading. Unless defined otherwise, all technical and scientific terms used herein have the same meaning as commonly understood by one of ordinary skill in the art.

When trade names are used herein, it is intended to independently include the tradename product and the active pharmaceutical ingredient(s) of the tradename product.

### Definitions

The following description sets forth exemplary methods, parameters and the like. It should be recognized, however, that such description is not intended as a limitation on the scope of the present disclosure but is instead provided as a description of exemplary embodiments.

As used in the present specification, the following words, phrases and symbols are generally intended to have the meanings as set forth below, except to the extent that the context in which they are used indicates otherwise.

The prefix “Cᵤ₋ᵥ” indicates that the following group has from u to v carbon atoms. For example, “C₁₋₈ alkyl” indicates that the alkyl group has from 1 to 8 carbon atoms.

“Isomers” are different compounds that have the same molecular formula. Isomers include stereoisomers, enantiomers and diastereomers.

A “stereoisomer” refers to a compound made up of the same atoms bonded by the same bonds but having different three-dimensional structures, which are not interchangeable.

“Enantiomers” are a pair of stereoisomers that are non-superimposable mirror images of each other. A 1:1 mixture of a pair of enantiomers is a “racemic” mixture. A mixture of enantiomers at a ratio other than 1:1 is a “scalemic” mixture.

“Diastereoisomers” are stereoisomers that have at least two asymmetric atoms, but which are not mirror-images of each other.

The absolute stereochemistry is specified according to the Cahn-Ingold-Prelog R-S system. When a compound is a pure enantiomer the stereochemistry at each chiral carbon may be specified by either R or S. Resolved compounds whose absolute configuration is unknown can be designated (+) or (−) depending on the direction (dextro- or levorotatory) which they rotate plane polarized light at the wavelength of the sodium D line. Certain of the compounds described herein contain one or more asymmetric centers and/or hindered rotation about a bond axis and may thus give rise to enantiomers, diastereomers, and other stereoisomeric forms that may be defined, in terms of absolute stereochemistry, as (R)- or (S)-. The present disclosure is meant to include all such possible isomers, including racemic mixtures, scalemic mixtures, diastereomeric mixtures, optically pure forms and intermediate mixtures. Optically active (R)- and (S)-isomers may be prepared using chiral synthons or chiral reagents, or resolved using conventional techniques.

Except as expressly defined otherwise, the present disclosure includes all tautomers of compounds detailed herein, even if only one tautomer is expressly represented (e.g., both tautomeric forms are intended and described by the presentation of one tautomeric form where a pair of two tautomers may exist). For example, if reference is made to a compound containing an amide (e.g., by structure or chemical name), it is understood that the corresponding imidic acid tautomer is included by this disclosure and described the same as if the amide were expressly recited either alone or together with the imidic acid. Where more than two tautomers may exist, the present disclosure includes all such tautomers even if only a single tautomeric form is depicted by chemical name and/or structure.

Compounds described herein may have chiral centers and/or geometric isomeric centers (E- and Z-isomers), and it is to be understood that all such optical, enantiomeric, diastereoisomeric and geometric isomers are encompassed. Where compounds are represented in their chiral form, it is understood that the embodiment encompasses, but is not limited to, the specific diastereomerically or enantiomerically enriched form. Where chirality is not specified but is present, it is understood that the embodiment is directed to either the specific diastereomerically or enantiomerically enriched form; or a racemic or scalemic mixture of such compound(s). As used herein, “scalemic mixture” is a mixture of stereoisomers at a ratio other than 1:1.

Compounds described herein may be in any morphological form such as, for example, crystalline or amorphous, as well as disordered crystals, liquid crystals, plastic crystals, mesophases, and the like, or any combination thereof.

Also provided are pharmaceutically acceptable hydrates, solvates, co-crystals, tautomeric forms, polymorphs, and prodrugs of the compounds described herein.

The term “hydrate” refers to the complex formed by the combining of a compound of Formula I, or any Formula disclosed herein, and water.

The term “solvate” refers to a complex formed by the combining of a compound of Formula I, or any other Formula as disclosed herein, and a solvent or a crystalline solid containing amounts of a solvent incorporated within the crystal structure. As used herein, the term “solvate” includes hydrates.

The term “co-crystal” refers to a crystalline material formed by combining a compound of Formula I, or any Formula disclosed herein and one or more co-crystal formers (i.e., a molecule, ion or atom). In certain instances, co-crystals may have improved properties as compared to the parent form (i.e., the free molecule, zwitterion, etc.) or a salt of the parent compound. Improved properties can be increased solubility, increased dissolution, increased bioavailability, increased dose response, decreased hygroscopicity, a crystalline form of a normally amorphous compound, a crystalline form of a difficult to salt or unsaltable compound, decreased form diversity, more desired morphology, and the like. Methods for making and characterizing co-crystals are known to those of skill in the art.

Any formula or structure given herein, including Formula I, or any Formula disclosed herein, is also intended to represent unlabeled forms as well as isotopically labeled forms of the compounds. Isotopically labeled compounds have structures depicted by the formulas given herein except that one or more atoms are replaced by an atom having a selected atomic mass or mass number. Examples of isotopes that can be incorporated into compounds of the disclosure include isotopes of hydrogen, carbon, nitrogen, oxygen, phosphorous, fluorine and chlorine, such as, but not limited to ²H (deuterium, D), ³H (tritium), ¹¹C, ¹³C, ¹⁴C, ¹⁵N, ¹⁸F, ³¹P, ³²P, ³⁵S, ³⁶Cl and ¹²⁵I. Various isotopically labeled compounds of the present disclosure, for example those into which radioactive isotopes such as ³H, ¹³C, and ¹⁴C are incorporated, may be useful to help determine or measure the effectiveness of the compounds by characterizing, for example, the site or mode of action, or binding affinity to pharmacologically important site of action. Such isotopically labeled compounds may be useful in metabolic studies, reaction kinetic studies, detection or imaging techniques, such as positron emission tomography (PET) or single-photon emission computed tomography (SPECT) including drug or substrate tissue distribution assays or in radioactive treatment of patients.

The disclosure also includes compounds of Formula I, or any Formula disclosed herein, in which from 1 to “n” hydrogens attached to a carbon atom is/are replaced by deuterium, in which n is the number of hydrogens in the molecule. Such compounds exhibit increased resistance to metabolism and are thus useful for increasing the half-life of any compound of Formula I when administered to a mammal. See, for example, Foster, “Deuterium Isotope Effects in Studies of Drug Metabolism”, Trends Pharmacol. Sci. 5(12): 524-527 (1984). Such compounds are synthesized by means well known in the art, for example by employing starting materials in which one or more hydrogen atoms have been replaced by deuterium.

Deuterium labeled or substituted therapeutic compounds of the disclosure may have improved DMPK (drug metabolism and pharmacokinetics) properties, relating to distribution, metabolism and excretion (ADME). Substitution with heavier isotopes such as deuterium may afford certain therapeutic advantages resulting from greater metabolic stability, for example increased in vivo half-life or reduced dosage requirements. An ¹⁸F labeled compound may be useful for PET or SPECT studies. Isotopically labeled compounds of this disclosure and prodrugs thereof can generally be prepared by carrying out the procedures disclosed in the schemes or in the examples and preparations described below by substituting a readily available isotopically labeled reagent for a non-isotopically labeled reagent. Further, substitution with heavier isotopes, particularly deuterium (i.e., ²H or D) may afford certain therapeutic advantages resulting from greater metabolic stability, for example increased in vivo half-life or reduced dosage requirements or an improvement in therapeutic index. It is understood that deuterium in this context is regarded as a substituent in the compound of the Formula I, or any Formula disclosed herein.

The concentration of such a heavier isotope, specifically deuterium, may be defined by an isotopic enrichment factor. In the compounds of this disclosure any atom not specifically designated as a particular isotope is meant to represent any stable isotope of that atom. Unless otherwise stated, when a position is designated specifically as “H” or “hydrogen,” the position is understood to have hydrogen at its natural abundance isotopic composition. Accordingly, in the compounds of this disclosure any atom specifically designated as a deuterium (D) is meant to represent deuterium.

The modifier “about” used in connection with a quantity is inclusive of the stated value and has the meaning dictated by the context (e.g., includes the degree of error associated with measurement of the particular quantity).

A “coformer” as used herein refers to a protic acid that is capable of forming a salt or co-crystal with a compound.

The term “coupling agent” as used herein refers to a reagent used to couple an amine and a carboxylic acid to form an amide. Non-limiting examples of coupling agents include n-propyl phosphonic anhydride, oxalyl chloride, thionyl chloride, phosgene, triphosgene, propanephosphonic acid anhydride, acetic anhydride, pivalic anhydride, benzoic anhydride, 2,4,6-trichlorobenzoyl chloride, dicyclohexylcarbodiimide, diisopropylcarbodiimide, 1-ethyl-3-(3-dimethylaminopropyl)carbodiimide (EDC), 1,1′-carbonyldiimidazole, ethyl chloroformate, isobutyl chloroformate, 2-(7-aza-1H-benzotriazole-1-yl)-1,1,3,3-tetramethyluronium hexafluorophosphate (HATU), O-benzotriazole-N,N,N′,N′-tetramethyluronium-hexafluoro-phosphate (HBTU), O-(7-azabenzotriazole-1-yl)-N,N,N′,N′-tetramethyluronium tetrafluoroborate (TATU), O-(benzotriazol-1-yl)-N,N,N′,N′-tetramethyluronium tetrafluoroborate (TBTU), O-(6-chlorobenzotriazol-1-yl)-N,N,N′,N′-tetramethyluronium hexafluorophosphate (HCTU), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (1-cyano-2-ethoxy-2-oxoethylidenaminooxy)dimethylamino-morpholino-carbenium hexafluorophosphate (COMU), and the like.

A “peptide coupling agent” includes, but is not limited to, 2-(7-aza-1H-benzotriazole-1-yl)-1,1,3,3-tetramethyluronium hexafluorophosphate (HATU), O-benzotriazole-N,N,N′,N′-tetramethyluronium-hexafluoro-phosphate (HBTU), O-(7-azabenzotriazole-1-yl)-N,N,N′,N′-tetramethyluronium tetrafluoroborate (TATU), O-(benzotriazol-1-yl)-N,N,N′,N′-tetramethyluronium tetrafluoroborate (TBTU), O-(6-chlorobenzotriazol-1-yl)-N,N,N′,N′-tetramethyluronium hexafluorophosphate (HCTU), (benzotriazol-1-yloxy)tris(dimethylamino)phosphonium hexafluorophosphate (BOP), (benzotriazol-1-yloxy)tripyrrolidinophosphonium hexafluorophosphate (PyBOP), (1-cyano-2-ethoxy-2-oxoethylidenaminooxy)dimethylamino-morpholino-carbenium hexafluorophosphate (COMU), and the like.

As used herein, the term “first promoter” refers to an acid useful in the conversion of a compound of Formula F to a compound of Formula A. Non-limiting examples of first promoters include carboxylic acids, sulfonic acids, halogenated alcohols, Lewis acids, inorganic acids, and combinations thereof.

As used herein, the term “second promoter” refers to a dehydrating agent useful in the ketalization of a compound of Formula J. Non-limiting examples of second promoters include trimethyl orthoformate, triethyl orthoformate, orthoacetates, molecular sieves, magnesium sulfate, sodium sulfate, azeotropic distillation, and combinations thereof.

The term “dicarbonyl reagent” as used herein refers to a reagent containing vicinal carbonyl groups useful in the conversion of a compound of Formula F to a compound of Formula A. Non-limiting examples of dicarbonyl reagents include dimethyl oxalate, diethyl oxalate, oxalyl chloride, chlorooxoacetates, and combinations thereof.

The term “phase transfer catalyst” as used herein refers to a substance that facilitates the transfer of ionic compounds into an organic phase from an aqueous phase or from a solid phase. A phase transfer catalyst facilitates the reaction between water-soluble and water-insoluble reaction components. In some embodiments of this invention, the phase transfer catalyst is selected from the group consisting of crown ethers, ammonium salts, phosphonium salts, cryptands, polyalkylene glycols, and mixtures and derivatives thereof. The phase transfer catalyst can be ionic or neutral. Exemplary ammonium salts include tetrabutylammonium chloride, tetrabutylammonium bromide, tetrabutylammonium hydroxide, tetrabutylammonium hydrogen sulfate, benzyltributylammonium bromide, 1-methylimidazolium hydrogen sulfate, tetramethylammonium chloride, tetramethylammonium bromide, benzyltriethylammonium chloride, methyltri-n-octylammonium chloride (also known as Aliquat™ 336), dodecyltrimethylammonium bromide, and mixtures thereof. Exemplary phosphonium salts include tetrabutylphosphonium chloride, tetraphenylphosphonium bromide, tetraphenylphosphonium chloride, triphenylmethylphosphonium bromide, triphenylmethylphosphonium chloride, and mixtures thereof.

“Alkyl” refers to an unbranched or branched saturated hydrocarbon chain. As used herein, alkyl has 1 to 20 carbon atoms (i.e., C₁₋₂₀ alkyl), 1 to 12 carbon atoms (i.e., C₁₋₁₂ alkyl), 1 to 8 carbon atoms (i.e., C₁₋₈ alkyl), 1 to 6 carbon atoms (i.e., C₁₋₆ alkyl), 1 to 4 carbon atoms (i.e., C₁₋₄ alkyl), 1 to 3 carbon atoms (i.e., C₁₋₃ alkyl), or 1 to 2 carbon atoms (i.e., C₁₋₂ alkyl). Examples of alkyl groups include, but are not limited to, methyl, ethyl, propyl, isopropyl, n-butyl, sec-butyl, iso-butyl, tert-butyl, pentyl, 2-pentyl, isopentyl, neopentyl, hexyl, 2-hexyl, 3-hexyl, and 3-methylpentyl. When an alkyl residue having a specific number of carbons is named by chemical name or identified by molecular formula, all positional isomers having that number of carbons may be encompassed; thus, for example, “butyl” includes n-butyl (i.e. —(CH₂)₃CH₃), sec-butyl (i.e. —CH(CH₃)CH₂CH₃), isobutyl (i.e. —CH₂CH(CH₃)₂) and tert-butyl (i.e. —C(CH₃)₃); and “propyl” includes n-propyl (i.e. —(CH₂)₂CH₃) and isopropyl (i.e. —CH(CH₃)₂).

“Aryl” refers to an aromatic carbocyclic group having a single ring (e.g., monocyclic) or multiple rings (e.g., bicyclic or tricyclic), including fused systems. As used herein, aryl has 6 to 20 ring carbon atoms (i.e., C₆₋₂₀ aryl), 6 to 12 carbon ring atoms (i.e., C₆₋₁₂ aryl), or 6 to 10 carbon ring atoms (i.e., C₆₋₁₀ aryl). Examples of aryl groups include, but are not limited to, phenyl, naphthyl, fluorenyl, and anthryl.

“Alkylene” refers to an unbranched and branched divalent hydrocarbon chain. As used herein, alkylene has 1 to 4 carbon atoms (i.e., C₁₋₄ alkylene). Examples of alkylene groups include, but are not limited to, —CH₂—, —CH₂CH₂—, —CH₂CH₂CH₂—, —CH₂CH(CH₃)—, —CH₂CH₂CH₂CH₂—, —CH₂CH(CH₂CH₃), —CH₂CH₂CH(CH₃)—, —CH₂CH(CH₃)CH₂—, and —C(CH₃)₂CH₂—.

The term “halo” or “halogen” includes fluoro, chloro, bromo and iodo.

“Haloalkyl” refers to an unbranched or branched alkyl group as defined above, wherein one or more hydrogen atoms are replaced by a halogen. For example, where a residue is substituted with more than one halogen, it may be referred to by using a prefix corresponding to the number of halogen moieties attached. Dihaloalkyl and trihaloalkyl refer to alkyl substituted with two (“di”) or three (“tri”) halo groups, which may be, but are not necessarily, the same halogen. Examples of haloalkyl include difluoromethyl (—CHF₂) and trifluoromethyl (—CF₃).

“Aryl-alkylene” refers to an alkylene group as defined herein which is bonded to an aryl group as defined herein. Accordingly, the point of attachment of an arylalkylene group is the alkylene group. The alkylene group of the “aryl-alkylene” is typically 1 to 4 carbon atoms (i.e., aryl-C₁₋₄ alkylene). Aryl-alkylene groups include, but are not limited to, benzyl (i.e., phenyl-CH₂—), 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl, and the like. The aryl-alkylene group typically comprises 6 to 18 carbon atoms, e.g., the alkylene moiety of the arylalkylene group is one to four carbon atoms and the aryl moiety is five to fourteen carbon atoms.

“Hydroxyl” and “hydroxy” are used interchangeably and refer to —OH. “Oxo” refers to the group (═O) or (O). Where tautomeric forms of the compound exist, hydroxyl and oxo groups are interchangeable.

The term “fused” refers to a ring structure described herein containing carbocyclic, heterocyclic, aromatic, and/or heteroaromatic rings that are connected via two adjacent atoms. For example, the bicyclic compounds depicted below incorporate a cyclopropane fused to a cyclohexane, a pyrrolidine fused to a benzene, and a thiophene fused to a furan, respectively:

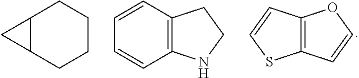

The term “bridged” refers to a carbocyclic or heterocyclic ring structure described herein containing two non-adjacent atoms in a ring that are connected via a single atom or a divalent group. Exemplary bridged bicyclic compounds are bicyclo[2.2.1]heptane and 1,4-diazabicyclo[2.2.2]octane, depicted below:

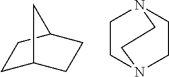

The term “spiro” refers to a ring structure described herein which has two ring structures, each of which may be carbocyclic or heterocyclic, that are connected via a single, shared atom (which atom is denoted a spiro atom). Accordingly, “spirocyclo” refers to a spiro cycloalkyl radical, or a spiro heterocycloalkyl radical.

The term “protecting group” refers to a moiety of a compound that masks or alters the properties of a functional group or the properties of the compound as a whole. Chemical protecting groups and strategies for protection/deprotection are well known in the art. See, e.g., Protective Groups in Organic Chemistry, Theodora W. Greene, John Wiley & Sons, Inc., New York, 1991. Protecting groups are often utilized to mask the reactivity of certain functional groups, to assist in the efficiency of desired chemical reactions, e.g., making and breaking chemical bonds in an ordered and planned fashion. The term “deprotecting” refers to removing the protecting group. The term “deprotecting agent” refers to a reagent useful for removing a protecting group from a compound.

The term “hydroxyl protecting group” refers to a moiety that masks or alters the properties of a hydroxyl group. Exemplary hydroxyl protecting groups include, but are not limited to, methyl, t-butyl, t-butoxymethyl, methoxymethyl, tetrahydropyranyl, 1-ethoxyethyl, 1-(2-chloroethoxy)ethyl, 2-trimethylsilylethyl, p-chlorophenyl, 2,4-dinitrophenyl, benzyl, 2,6-dichlorobenzyl, diphenylmethyl, p-nitrobenzyl, triphenylmethyl, trimethylsilyl, triethylsilyl, t-butyldimethylsilyl, t-butyl-diphenylsilyl (TBDPS), triphenylsilyl, benzoylformate, acetate, chloroacetate, trichloroacetate, trifluoroacetate, pivaloate, benzoate, p-phenylbenzoate, 9-fluorenylmethyl carbonate, mesylate, and tosylate.

### Abbreviations

Abbreviations as used herein have respective meanings as follows:
aq. aqueous
Bn benzyl
BOP (benzotriazol-1-yloxy)tris(dimethylamino)phosphonium hexafluorophosphate
br broad
CDI carbonyldiimidazole
COMU or COMU® (1-cyano-2-ethoxy-2-oxoethylidenaminooxy)dimethylamino-morpholino-carbenium hexafluorophosphate
d doublet
DBU 1,8-diazabicyclo[5.4.0]undec-7-ene
DCM dichloromethane
dd doublet of doublets
ddd doublet of doublet of doublets
DEO diethyl oxalate
DMA N,N-dimethylacetamide
DMF dimethylformamide
DMO dimethyl oxalate
DMSO dimethylsulfoxide
dt doublet of triplets
Et ethyl
EtOH ethanol
h hour(s)
HATU 2-(7-aza-1H-benzotriazole-1-yl)-1,1,3,3-tetramethyluronium hexafluorophosphate
HBTU O-benzotriazole-N,N,N′,N′-tetramethyluronium-hexafluoro-phosphate
HCTU O-(6-chlorobenzotriazol-1-yl)-N,N,N′,N′-tetramethyluronium hexafluorophosphate
HOAt 1-hydroxy-7-azabenzotriazole
HOBt 1-hydroxybenzotriazole
Hz hertz
J coupling constant
m multiplet
M Molar
MTBE methyl tert-butyl ether
Me methyl
MeOH methanol
MeTHF 2-methyltetrahydrofuran
MHz megahertz
min. minute(s)
MS mass spectroscopy
MTBE methyl-tert-butyl ether
m/z Mass to charge
N normal
NaOMe sodium methoxide
NMR nuclear magnetic resonance
NMT not more than
PG protecting group
PyBOP (benzotriazol-1-yloxy)tripyrrolidinophosphonium hexafluorophosphate
q quartet
s singlet
t triplet
TATU O-(7-azabenzotriazole-1-yl)-N,N,N′,N′-tetramethyluronium tetrafluoroborate
TBTU O-(benzotriazol-1-yl)-N,N,N′,N′-tetramethyluronium tetrafluoroborate
TEA triethylamine
TFA trifluoroacetic acid
TFE 2,2,2-trifluoroethanol
tol toluene
TsOH or p-TsOH p-toluenesulfonic acid
wt % weight percent

### Methods and Intermediates

Except as otherwise noted, the methods and techniques of the present disclosure are generally performed according to conventional methods well known in the art and as described in various general and more specific references that are cited and discussed throughout the present specification. See, e.g., Loudon, Organic Chemistry, 5^{th} edition, New York: Oxford University Press, 2009; Smith, March's Advanced Organic Chemistry: Reactions, Mechanisms, and Structure, 7^{th} edition, Wiley-Interscience, 2013.

In certain instances, the processes disclosed herein involve a step of forming a salt of a compound of the present disclosure.

Compounds as described herein can be purified by any of the means known in the art, including chromatographic means, such as high performance liquid chromatography (HPLC), preparative thin layer chromatography, flash column chromatography, supercritical fluid chromatography (SFC), and ion exchange chromatography. Any suitable stationary phase can be used, including normal and reversed phases as well as ionic resins. Most typically the disclosed compounds are purified via silica gel and/or alumina chromatography. See, e.g., Introduction to Modern Liquid Chromatography, 2^{nd} ed., ed. L. R. Snyder and J. J. Kirkland, John Wiley and Sons, 1979; and Thin Layer Chromatography, E. Stahl (ed.), Springer-Verlag, New York, 1969.

Exemplary chemical entities useful in methods of the embodiments will now be described by reference to illustrative synthetic schemes for their general preparation herein and the specific examples that follow. One of skill in the art will recognize that the transformations shown in the schemes below may be performed in any order that is compatible with the functionality of the particular pendant groups. In some embodiments, each of the reactions depicted in the general schemes is run at a temperature from about −80° C. to the reflux temperature of the organic solvent used.

The compounds disclosed herein may display atropisomerism resulting from steric hindrance affecting the axial rotation rate around a single bond. The resultant conformational isomers may each be observed as distinct entities by characterization techniques such as NMR and HPLC. The compounds disclosed herein may exist as a mixture of atropisomers. However, the detection of atropisomers is dependent on factors such as temperature, solvent, conditions of purification, and timescale of spectroscopic technique. The interconversion rate at room temperature has a half-life of minutes to hours, hours to days, or days to years. The ratio of atropisomers at equilibrium may not be unity. Characterization data presented herein may not represent the equilibrium state depending on the conditions of isolation and characterization which may include but not limited to handling, solvents used, and temperature.

The present disclosure provides in some embodiments processes and intermediates for preparing the compound of Formula I and salts thereof. In other embodiments, the disclosure provides processes for preparing intermediates that can be used to prepare the compound of Formula I and salts thereof.

A method of preparing a compound of Formula I:

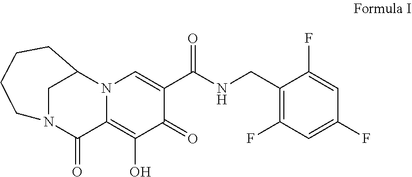 [SMILES: O=C(NCc1c(F)cc(F)cc1F)c1cn2c(c(O)c1=O)C(=O)N1CCCCC2C1]

or a salt thereof is provided, comprising treating a compound of Formula A:

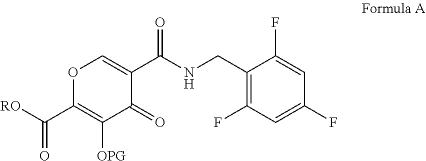

with a compound of Formula B:

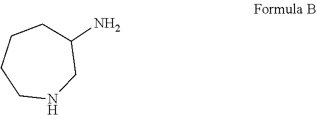

or a salt thereof, wherein PG is a hydroxyl protecting group and R is C₁₋₆ alkyl, C₆₋₁₀ aryl-C₁₋₄ alkylene, or C₆₋₁₀ aryl.

In some embodiments, the compound of Formula A is treated with the compound of Formula B or a salt thereof in the presence of a first solvent.

In some embodiments, the first solvent is an alcohol, an ester, an ether, a halogenated solvent, an aliphatic hydrocarbon, an aromatic hydrocarbon, a nitrile, a polar aprotic solvent, or a combination thereof. In some embodiments, the first solvent is an organic alcohol. In some embodiments, the first solvent is methanol, ethanol, 2-propanol, or a combination thereof. In some embodiments, the first solvent is an organic ester. In some embodiments, the first solvent is ethyl acetate, isopropyl acetate, or a combination thereof. In some embodiments, the first solvent is an organic ether. In some embodiments, the first solvent is, tetrahydrofuran, 2-methyltetrahydrofuran, tert-butyl methyl ether, or a combination thereof. In some embodiments, the first solvent is an organic halogenated solvent. In some embodiments, the first solvent is dichloromethane, 1,2-dichloroethane, chlorobenzene, or a combination thereof. In some embodiments, the first solvent is an organic aliphatic hydrocarbon. In some embodiments, the first solvent is heptane (e.g., n-heptane). In some embodiments, the first solvent is an organic aromatic hydrocarbon. In some embodiments, the first solvent is benzene, toluene, or a combination thereof. In some embodiments, the first solvent is an organic nitrile. In some embodiments, the first solvent is acetonitrile. In some embodiments, the first solvent is a polar aprotic solvent. In some embodiments, the first solvent is N,N-dimethylformamide, N,N-dimethylacetamide, N-methyl-2-pyrrolidone, or a combination thereof. In some embodiments, the first solvent is methanol, ethanol, 2-propanol, ethyl acetate, isopropyl acetate, tetrahydrofuran, 2-methyltetrahydrofuran, tert-butyl methyl ether, dichloromethane, 1,2-dichloroethane, chlorobenzene, benzene, toluene, n-heptane, acetonitrile, N,N-dimethylformamide, N,N-dimethylacetamide, N-methyl-2-pyrrolidone, or a combination thereof. In some embodiments, the first solvent is methanol, toluene, or a combination thereof. In some embodiments, the first solvent is toluene. In some embodiments, the first solvent is methanol. In some embodiments the first solvent is a combination of toluene and methanol. In some embodiments, the first solvent is toluene-methanol in a ratio of 10:1 or 9:1 or 8:1 or 7:1 or 6:1 or 5:1 or 4:1 or 3:1 or 2:1 or 1:1. In some embodiments, the first solvent is ethanol, heptane, or a combination thereof. In some embodiments, the first solvent is a combination of ethanol and heptane.

In some embodiments, the compound of Formula A is treated with the compound of Formula B or a salt thereof at a temperature of about 20 to about 120° C. In some embodiments, the compound of Formula A is treated with the compound of Formula B or a salt thereof at a temperature of about 45 to about 65° C. In some embodiments, the compound of Formula A is treated with the compound of Formula B or a salt thereof at a temperature of about 55° C. In some embodiments, the compound of Formula A is treated with the compound of Formula B or a salt thereof at a temperature of about 40 to about 60° C. In some embodiments, the compound of Formula A is treated with the compound of Formula B or a salt thereof at a temperature of about 50° C. In some embodiments, the compound of Formula A is treated with the compound of Formula B or a salt thereof at a temperature of about 70° C.

In some embodiments, the first solvent is a combination of toluene and methanol and the temperature is about 45 to about 65° C. In some embodiments, the first solvent is a combination of toluene and methanol and the temperature is about 55° C.

In some embodiments, the first solvent is methanol and the temperature is about 40 to about 60° C. In some embodiments, the first solvent is methanol and the temperature is about 40 to about 50° C.

In some embodiments, the first solvent is a combination of ethanol and heptane and the temperature is about 40 to about 60° C. In some embodiments, the first solvent is a combination of ethanol and heptane and the temperature is about 70° C.

In some embodiments, the compound of Formula A is treated with the compound of Formula B or a salt thereof in the presence of a first base.

In some embodiments, the first base is a carbonate, a bicarbonate, a metal hydride, an alkoxide, a hydroxide, a phosphate, an amine, a basic aromatic compound, an acetate, a formate, or a combination thereof. In some embodiments, the first base is lithium carbonate, sodium carbonate, potassium carbonate, cesium carbonate, lithium bicarbonate, sodium bicarbonate, potassium bicarbonate, lithium hydride, sodium hydride, potassium hydride, sodium methoxide, sodium tert-butoxide, lithium tert-butoxide, sodium hydroxide, potassium hydroxide, sodium phosphate dibasic, sodium phosphate tribasic, potassium phosphate dibasic, potassium phosphate tribasic, lithium phosphate dibasic, lithium phosphate tribasic, lithium hydroxide, lithium methoxide, potassium methoxide, potassium tert-butoxide, lithium phosphate dibasic, lithium phosphate tribasic, triethylamine, diisopropylethylamine, 1,4-diazabicylo[2.2.2]-octane, 1,8-diazabicyclo[5.4.0]undec-7-ene, pyridine, 2,6-lutidine, imidazole, N-methylmorpholine, N-ethylmorpholine, lithium acetate, sodium acetate, potassium acetate, lithium formate, sodium formate, potassium formate, or a combination thereof. In some embodiments, the first base is a carbonate. In some embodiments, the first base is lithium carbonate, sodium carbonate, potassium carbonate, cesium carbonate, or a combination thereof. In some embodiments, the first base is a bicarbonate. In some embodiments, the first base is lithium bicarbonate, sodium bicarbonate, potassium bicarbonate, or a combination thereof. In some embodiments, the first base is a metal hydride. In some embodiments, the first base is lithium hydride, sodium hydride, potassium hydride, or a combination thereof. In some embodiments, the first base is an alkoxide. In some embodiments, the first base is sodium methoxide, sodium tert-butoxide, lithium tert-butoxide, or a combination thereof. In some embodiments, the first base is a hydroxide. In some embodiments, the first base is sodium hydroxide, potassium hydroxide, or a combination thereof. In some embodiments, the first base is a phosphate. In some embodiments, the first base is sodium phosphate dibasic, sodium phosphate tribasic, potassium phosphate dibasic, potassium phosphate tribasic, or a combination thereof. In some embodiments, the first base is an amine. In some embodiments, the first base is triethylamine, diisopropylethylamine, 1,4-diazabicylo[2.2.2]-octane, 1,8-diazabicyclo[5.4.0]undec-7-ene, or a combination thereof. In some embodiments, the first base is a basic aromatic compound. In some embodiments, the first base is pyridine, 2,6-lutidine, imidazole, N-methylmorpholine, N-ethylmorpholine, or a combination thereof. In some embodiments, the first base is an acetate. In some embodiments, the first base is lithium acetate, sodium acetate, potassium acetate, or a combination thereof. In some embodiments, the first base is a formate. In some embodiments, the first base is lithium formate, sodium formate, potassium formate, or a combination thereof.

In some embodiments, the compound of Formula A is treated with the compound of Formula B or a salt thereof in absence of any base.

In some embodiments, the first solvent is a combination of toluene and methanol, the temperature is about 45 to about 65° C., and Formula A is treated with the compound of Formula B or a salt thereof in absence of the first base. In some embodiments, the first solvent is a combination of toluene and methanol and the temperature is about 55° C., and Formula A is treated with the compound of Formula B or a salt thereof in absence of the first base.

In some embodiments, the compound of Formula B is a salt, comprising a coformer.

In some embodiments, the coformer is an inorganic acid or an organic acid. In some embodiments, the coformer is a carboxylic acid or a sulfonic acid. In some embodiments, the coformer is sulfuric acid, hydrochloric acid, hydrobromic acid, acetic acid, pivalic acid, trifluoroacetic acid, phosphoric acid, methanesulfonic acid, p-toluenesulfonic acid, mandelic acid, tartaric acid, citric acid, benzenesulfonic acid, vanillic acid, formic acid, oxalic acid, fumaric acid, maleic acid, malic acid, succinic acid, cinnamic acid, or a combination thereof. In some embodiments, the coformer is sulfuric acid, hydrochloric acid, methanesulfonic acid, p-toluenesulfonic acid, mandelic acid, tartaric acid, citric acid, or a combination thereof. In some embodiments, the coformer is sulfuric acid, hydrochloric acid, hydrobromic acid, acetic acid, pivalic acid, trifluoroacetic acid, phosphoric acid, methanesulfonic acid, p-toluenesulfonic acid, or a combination thereof. In some embodiments, the coformer is an inorganic acid. In some embodiments, the coformer is sulfuric acid, hydrochloric acid, hydrobromic acid, phosphoric acid, or a combination thereof. In some embodiments, the coformer is an organic acid where, in some embodiments, the organic acid is a carboxylic acid, a sulfonic acid, or a combination thereof. In some embodiments, the coformer is a carboxylic acid. In some embodiments, the coformer is acetic acid, pivalic acid, trifluoroacetic acid, mandelic acid, tartaric acid, citric acid, vanillic acid, formic acid, oxalic acid, fumaric acid, maleic acid, malic acid, succinic acid, cinnamic acid, or a combination thereof. In some embodiments, the coformer is a sulfonic acid. In some embodiments, the coformer is methanesulfonic acid, p-toluenesulfonic acid, benzenesulfonic acid or a combination thereof.

In some embodiments, the treatment of the compound of Formula A with a compound of Formula B or a salt thereof provides a compound of Formula C:

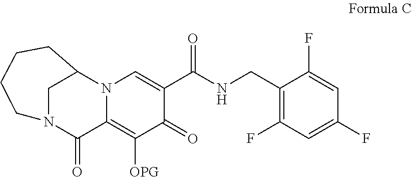

wherein deprotection of the compound of Formula C provides a compound of Formula I or a salt thereof. In some embodiments, the compound of Formula C is a compound of Formula C-A and the compound of Formula I is a compound of Formula I-A:

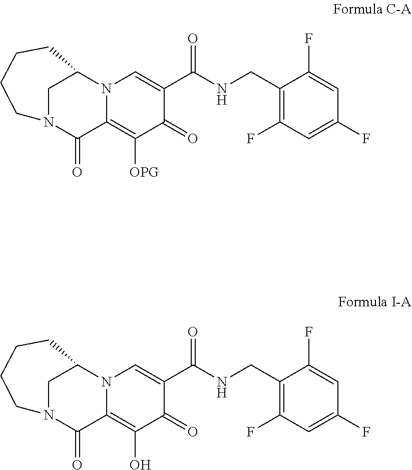

or a salt thereof.

In some embodiments, the deprotection of the compound of Formula C comprises treatment of the compound of Formula C with a deprotecting agent. Any suitable deprotecting agent can be used for the deprotection of the compound of Formula C. Suitable deprotecting agents include Lewis acids, Bronsted acids (e.g., acids such as sulfuric acid, hydrochloric acid, hydrobromic acid, carboxylic acids, phosphoric acid, sulfonic acids), Lewis bases (e.g, tetrabutylammonium fluoride), Bronsted bases, transition metal catalysts (e.g., palladium, nickel), and oxidants (e.g., dichlorodicyanoquinone).

In some embodiments, the deprotecting agent is an acid. In some embodiments, the deprotecting agent is sulfuric acid, hydrochloric acid, hydrobromic acid, a carboxylic acid, phosphoric acid, a sulfonic acid, or a combination thereof. In some embodiments, the deprotecting agent is a carboxylic acid. In some embodiments, the deprotecting agent is, acetic acid, pivalic acid, trifluoroacetic acid, or a combination thereof. In some embodiments, the deprotecting agent is trifluoroacetic acid. In some embodiments, the deprotecting agent is a sulfonic acid. In some embodiments, the deprotecting agent is methanesulfonic acid, p-toluenesulfonic acid, or a combination thereof.

In some embodiments, the deprotecting agent is a metal halide, an organoborate, a metal trimethylsilanoate, sodium ethanethiolate, sodium hexamethyldisiloxane, or a metal triflate. In some embodiments, the deprotecting agent is lithium chloride, magnesium chloride, aluminum chloride, lithium bromide, magnesium bromide, aluminum bromide, lithium iodide, sodium iodide, potassium iodide, boron trifluoride diethyl etherate, boron trifluoride dimethyl etherate, boron tribromide, boron trichloride, trimethylborate, triethylborate, chlorotrimethylsilane, iodotrimethylsilane, lithium trimethylsilanoate, sodium trimethylsilanoate, potassium trimethylsilanoate, lithium triflate, magnesium triflate, aluminum triflate, or a combination thereof. In some embodiments, the deprotecting agent is boron trichloride, lithium bromide, magnesium chloride, aluminum chloride, or a combination thereof. In some embodiments, the deprotecting agent is lithium chloride. In some embodiments, the deprotecting agent is a metal halide. In some embodiments, the deprotecting agent is lithium chloride, magnesium chloride, aluminum chloride, lithium bromide, magnesium bromide, aluminum bromide, lithium iodide, sodium iodide, potassium iodide, boron trifluoride diethyl etherate, boron trifluoride dimethyl etherate, boron tribromide, boron trichloride, chlorotrimethylsilane, iodotrimethylsilane, or a combination thereof. In some embodiments, the deprotecting agent is an organoborate. In some embodiments, the deprotecting agent is trimethylborate, triethylborate, or a combination thereof. In some embodiments, the deprotecting agent is a metal trimethylsilanoate. In some embodiments, the deprotecting agent is lithium trimethylsilanoate, sodium trimethylsilanoate, potassium trimethylsilanoate, or a combination thereof. In some embodiments, the deprotecting agent is a metal triflate. In some embodiments, the deprotecting agent is lithium triflate, magnesium triflate, aluminum triflate, or a combination thereof.

In some embodiments, the second solvent is an alcohol, an ester, an ether, a halogenated solvent, an aliphatic hydrocarbon, an aromatic hydrocarbon, a nitrile, a polar aprotic solvent, or a combination thereof. In some embodiments, the second solvent is methanol, ethanol, 2-propanol, ethyl acetate, isopropyl acetate, tetrahydrofuran, 2-methyltetrahydrofuran, tert-butyl methyl ether, dichloromethane, 1,2-dichloroethane, chlorobenzene, benzene, toluene, n-heptane, acetonitrile, N,N-dimethylformamide, N,N-dimethylacetamide, N-methyl-2-pyrrolidone, or a combination thereof. In some embodiments, the second solvent is N-methyl-2-pyrrolidone. In some embodiments, the second solvent is toluene. In some embodiments, the second solvent is an organic alcohol. In some embodiments, the second solvent is methanol, ethanol, 2-propanol, or a combination thereof. In some embodiments, the second solvent is an organic ester. In some embodiments, the second solvent is ethyl acetate, isopropyl acetate, or a combination thereof. In some embodiments, the second solvent is an organic ether. In some embodiments, the second solvent is tetrahydrofuran, 2-methyltetrahydrofuran, tert-butyl methyl ether, or a combination thereof. In some embodiments, the second solvent is an organic halogenated solvent. In some embodiments, the second solvent is dichloromethane, 1,2-dichloroethane, chlorobenzene, or a combination thereof. In some embodiments, the second solvent is an organic aliphatic hydrocarbon. In some embodiments, the second solvent is heptane (e.g., n-heptane). In some embodiments, the second solvent is an organic aromatic hydrocarbon. In some embodiments, the second solvent is benzene, toluene, or a combination thereof. In some embodiments, the second solvent is an organic nitrile. In some embodiments, the second solvent is acetonitrile. In some embodiments, the second solvent is a polar aprotic solvent. In some embodiments, the second solvent is N,N-dimethylformamide, N,N-dimethylacetamide, N-methyl-2-pyrrolidone, or a combination thereof.

In some embodiments, the deprotecting agent is a carboxylic acid and the second solvent is toluene. In some embodiments, the deprotecting agent is trifluoroacetic acid and the second solvent is toluene.

In some embodiments, the deprotection of the compound of Formula C is at a temperature of about 0 to about 120° C. In some embodiments, the deprotection of the compound of Formula C is at a temperature of about 45 to about 65° C. In some embodiments, the deprotection of the compound of Formula C is at a temperature of about 55° C. In some embodiments, the deprotection of the compound of Formula C is at a temperature of about 90 to about 110° C. In some embodiments, the deprotection of the compound of Formula C is at a temperature of about 100° C.

In some embodiments, the deprotecting agent is trifluoroacetic acid, the second solvent is toluene, and the temperature is about 45 to about 65° C. In some embodiments, the deprotecting agent is trifluoroacetic acid, the second solvent is toluene, and the temperature is about 55° C.

In some embodiments, the deprotecting agent is lithium chloride, the second solvent is N-methyl-2-pyrrolidone, and the temperature is about 90 to about 110° C. In some embodiments, the deprotecting agent is lithium chloride, the second solvent is N-methyl-2-pyrrolidone, and the temperature is about 100° C.

In some embodiments, the method further comprises preparing the compound of Formula A by reacting a compound of Formula D:

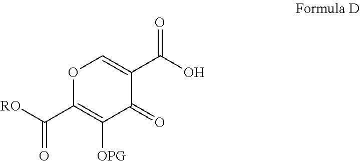

with a compound of Formula E:

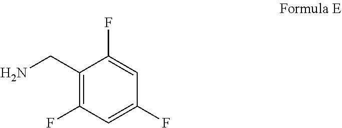 [SMILES: NCc1c(F)cc(F)cc1F]

or a salt thereof in the presence of a first coupling agent.

Any suitable coupling agent can be used for reacting the compound of Formula D with the compound of Formula E or a salt thereof. In some embodiments, the first coupling agent is oxalyl chloride, 1,1′-carbonyldiimidazole, thionyl chloride, phosgene, triphosgene, an alkyl chloroformate, a carbodiimide, propanephosphonic acid anhydride, a carboxylic anhydride, a carboxylic acid chloride, or a peptide coupling agent. In some embodiments, the first coupling agent is ethyl chloroformate or isobutyl chloroformate, oxalyl chloride, 1,1′-carbonyldiimidazole, thionyl chloride, phosgene, triphosgene, propanephosphonic acid anhydride, acetic anhydride, pivalic anhydride, benzoic anhydride, 2,4,6-trichlorobenzoyl chloride, dicyclohexylcarbodiimide, diisopropylcarbodiimide, 1-ethyl-3-(3-dimethylaminopropyl) carbodiimide, HATU, HBTU, TATU, TBTU, HCTU, BOP, PyBOP, COMU, or a combination thereof. In some embodiments, the first coupling agent is oxalyl chloride, 1,1′-carbonyldiimidazole, thionyl chloride, phosgene, triphosgene, or a combination thereof. In some embodiments, the first coupling agent is an alkyl chloroformate. In some embodiments, the first coupling agent is ethyl chloroformate, isobutyl chloroformate, or a combination thereof. In some embodiments, the first coupling agent is a carbodiimide. In some embodiments, the first coupling agent is dicyclohexylcarbodiimide, diisopropylcarbodiimide, 1-ethyl-3-(3-dimethylaminopropyl) carbodiimide, or a combination thereof. In some embodiments, the first coupling agent is propanephosphonic acid anhydride (T3P). In some embodiments, the first coupling agent is a peptide coupling agent. In some embodiments, the first coupling agent is HATU, HBTU, TATU, TBTU, HCTU, BOP, PyBOP, COMU, or a combination thereof. In some embodiments, the first coupling agent is oxalyl chloride.

In some embodiments, the compound of Formula D and the compound of Formula E or a salt thereof are reacted further in the presence of a first catalyst.

In some embodiments, the first catalyst is an amide, dimethylaminopyridine, 1-hydroxybenzotriazole, 1-hydroxy-7-azabenzotriazole, N,N-dimethylformamide, or a combination thereof. In some embodiments, the first catalyst is N,N-dimethylformamide, dimethylacetamide, N-methyl-2-pyrrolidone, dimethylaminopyridine, 1-hydroxybenzotriazole, 1-hydroxy-7-azabenzotriazole, or a combination thereof. In some embodiments, the first catalyst is an amide. In some embodiments, the first catalyst is N,N-dimethylformamide, dimethylacetamide, N-methyl-2-pyrrolidone, or a combination thereof. In some embodiments, the first catalyst is N,N-dimethylformamide.

In some embodiments, the first coupling agent is oxalyl chloride and the first catalyst is an amide. In some embodiments, the first coupling agent is oxalyl chloride and the first catalyst is N,N-dimethylformamide, dimethylacetamide, N-methyl-2-pyrrolidone, dimethylaminopyridine, 1-hydroxybenzotriazole, 1-hydroxy-7-azabenzotriazole, or a combination thereof. In some embodiments, the first coupling agent is oxalyl chloride and the first catalyst is N,N-dimethylformamide.

In some embodiments, the compound of Formula D and the compound of Formula E or a salt thereof are reacted further in the presence of a second base.

In some embodiments, the second base is a carbonate, a bicarbonate, a metal hydride, an alkoxide, a phosphate, an amine, a basic aromatic compound, or a combination thereof. In some embodiments, the second base is lithium carbonate, sodium carbonate, potassium carbonate, cesium carbonate, lithium bicarbonate, sodium bicarbonate, potassium bicarbonate, lithium hydride, sodium hydride, potassium hydride, sodium phosphate dibasic, sodium phosphate tribasic, potassium phosphate dibasic, potassium phosphate tribasic, lithium phosphate dibasic, lithium phosphate tribasic, lithium tert-butoxide, sodium tert-butoxide, potassium tert-butoxide, triethylamine, diisopropylethylamine, 4-methylmorpholine, pyridine, 2,6-lutidine, imidazole, or a combination thereof. In some embodiments, the second base is lithium carbonate, sodium carbonate, potassium carbonate, cesium carbonate, lithium bicarbonate, sodium bicarbonate, potassium bicarbonate, lithium hydride, sodium hydride, potassium hydride, sodium phosphate dibasic, sodium phosphate tribasic, potassium phosphate dibasic, potassium phosphate tribasic, triethylamine, diisopropylethylamine, 4-methylmorpholine, pyridine, 2,6-lutidine, imidazole, or a combination thereof. In some embodiments, the second base is a carbonate. In some embodiments, the second base is lithium carbonate, sodium carbonate, potassium carbonate, cesium carbonate, or a combination thereof. In some embodiments, the second base is a bicarbonate. In some embodiments, the second base is lithium bicarbonate, sodium bicarbonate, potassium bicarbonate, or a combination thereof. In some embodiments, the second base is a metal hydride. In some embodiments, the second base is lithium hydride, sodium hydride, potassium hydride, or a combination thereof. In some embodiments, the second base is an alkoxide. In some embodiments, the second base is lithium tert-butoxide, sodium tert-butoxide, potassium tert-butoxide, or a combination thereof. In some embodiments, the second base is a phosphate. In some embodiments, the second base is sodium phosphate dibasic, sodium phosphate tribasic, potassium phosphate dibasic, potassium phosphate tribasic, lithium phosphate dibasic, lithium phosphate tribasic, or a combination thereof. In some embodiments, the second base is an amine. In some embodiments, the second base is triethylamine, diisopropylethylamine, 4-methylmorpholine, or a combination thereof. In some embodiments, the second base is triethylamine. In some embodiments, the second base is a basic aromatic compound. In some embodiments, the second base is pyridine, 2,6-lutidine, imidazole, or a combination thereof.

In some embodiments, the first coupling agent is oxalyl chloride, the first catalyst is an amide, and the second base is an amine. In some embodiments, the first coupling agent is oxalyl chloride, the first catalyst is dimethylacetamide, N-methyl-2-pyrrolidone, dimethylaminopyridine, 1-hydroxybenzotriazole, 1-hydroxy-7-azabenzotriazole, N,N-dimethylformamide, or a combination thereof, and the second base is an amine. In some embodiments, the first coupling agent is oxalyl chloride, the first catalyst is N,N-dimethylformamide, and the second base is an amine.

In some embodiments, the first coupling agent is oxalyl chloride, the first catalyst is an amide, and the second base is triethylamine. In some embodiments, the first coupling agent is oxalyl chloride, the first catalyst is dimethylacetamide, N-methyl-2-pyrrolidone, dimethylaminopyridine, 1-hydroxybenzotriazole, 1-hydroxy-7-azabenzotriazole, N,N-dimethylformamide, or a combination thereof, and the second base is triethylamine. In some embodiments, the first coupling agent is oxalyl chloride, the first catalyst is N,N-dimethylformamide, and the second base is triethylamine.

In some embodiments, the compound of Formula D and the compound of Formula E or a salt thereof are reacted further in the presence of a third solvent.

In some embodiments, the third solvent is an ester, an ether, a halogenated solvent, an aliphatic hydrocarbon, an aromatic hydrocarbon, a nitrile, a polar aprotic solvent, or a combination thereof. In some embodiments, the third solvent is ethyl acetate, isopropyl acetate, tetrahydrofuran, 2-methyltetrahydrofuran, tert-butyl methyl ether, dichloromethane, 1,2-dichloroethane, chlorobenzene, benzene, toluene, n-heptane, acetonitrile, N,N-dimethylformamide, N,N-dimethylacetamide, N-methyl-2-pyrrolidone, or a combination thereof. In some embodiments, the third solvent is an organic ester. In some embodiments, the third solvent is ethyl acetate, isopropyl acetate, or a combination thereof. In some embodiments, the third solvent is an organic ether. In some embodiments, the third solvent is tetrahydrofuran, 2-methyltetrahydrofuran, tert-butyl methyl ether, or a combination thereof. In some embodiments, the third solvent is an organic halogenated solvent. In some embodiments, the third solvent is dichloromethane, 1,2-dichloroethane, chlorobenzene, or a combination thereof. In some embodiments, the third solvent is an organic aliphatic hydrocarbon. In some embodiments, the third solvent is heptane (e.g., n-heptane). In some embodiments, the third solvent is an organic aromatic hydrocarbon. In some embodiments, the third solvent is benzene, toluene, or a combination thereof. In some embodiments, the third solvent is an organic nitrile. In some embodiments, the third solvent is acetonitrile. In some embodiments, the third solvent is a polar aprotic solvent. In some embodiments, the third solvent is N,N-dimethylformamide, N,N-dimethylacetamide, N-methyl-2-pyrrolidone, or a combination thereof. In some embodiments, the third solvent is dichloromethane.

In some embodiments, the first coupling agent is oxalyl chloride, the first catalyst is an amide, the second base is an amine, and the third solvent is an organic halogenated solvent. In some embodiments, the first coupling agent is oxalyl chloride, the first catalyst is dimethylacetamide, N-methyl-2-pyrrolidone, dimethylaminopyridine, 1-hydroxybenzotriazole, 1-hydroxy-7-azabenzotriazole, N,N-dimethylformamide, or a combination thereof, the second base is an amine, and the third solvent is an organic halogenated solvent. In some embodiments, the first coupling agent is oxalyl chloride, the first catalyst is N,N-dimethylformamide, and the second base is an amine, and the third solvent is an organic halogenated solvent.

In some embodiments, the first coupling agent is oxalyl chloride, the first catalyst is an amide, the second base is triethylamine, and the third solvent is an organic halogenated solvent. In some embodiments, the first coupling agent is oxalyl chloride, the first catalyst is dimethylacetamide, N-methyl-2-pyrrolidone, dimethylaminopyridine, 1-hydroxybenzotriazole, 1-hydroxy-7-azabenzotriazole, N,N-dimethylformamide, or a combination thereof, the second base is triethylamine, and the third solvent is an organic halogenated solvent. In some embodiments, the first coupling agent is oxalyl chloride, the first catalyst is N,N-dimethylformamide, the second base is triethylamine, and the third solvent is an organic halogenated solvent.

In some embodiments, the first coupling agent is oxalyl chloride, the first catalyst is an amide, the second base is an amine, and the third solvent is dichloromethane. In some embodiments, the first coupling agent is oxalyl chloride, the first catalyst is dimethylacetamide, N-methyl-2-pyrrolidone, dimethylaminopyridine, 1-hydroxybenzotriazole, 1-hydroxy-7-azabenzotriazole, N,N-dimethylformamide, or a combination thereof, the second base is an amine, and the third solvent is dichloromethane. In some embodiments, the first coupling agent is oxalyl chloride, the first catalyst is N,N-dimethylformamide, the second base is an amine, and the third solvent is dichloromethane.

In some embodiments, the first coupling agent is oxalyl chloride, the first catalyst is an amide, the second base is triethylamine, and the third solvent is dichloromethane. In some embodiments, the first coupling agent is oxalyl chloride, the first catalyst is dimethylacetamide, N-methyl-2-pyrrolidone, dimethylaminopyridine, 1-hydroxybenzotriazole, 1-hydroxy-7-azabenzotriazole, N,N-dimethylformamide, or a combination thereof, the second base is triethylamine, and the third solvent is dichloromethane. In some embodiments, the first coupling agent is oxalyl chloride, the first catalyst is N,N-dimethylformamide, the second base is triethylamine, and the third solvent is dichloromethane.

In some embodiments, the compound of Formula D and the compound of Formula E or a salt thereof are reacted at a temperature of about −30 to about 60° C. In some embodiments, the compound of Formula D and the compound of Formula E or a salt thereof are reacted at a temperature of about −20 to about 10° C. In some embodiments, the compound of Formula D and the compound of Formula E or a salt thereof are reacted at a temperature of about −10° C.

In some embodiments, the first coupling agent is oxalyl chloride, the first catalyst is an amide, the second base is an amine, the third solvent is an organic halogenated solvent, and the temperature is about −20 to about 10° C. In some embodiments, the first coupling agent is oxalyl chloride, the first catalyst an amide, the second base is an amine, the third solvent is an organic halogenated solvent, and the temperature is about −10° C. In some embodiments, the first coupling agent is oxalyl chloride, the first catalyst is N,N-dimethylformamide, the second base is an amine, the third solvent is an organic halogenated solvent, and the temperature is about −20 to about 10° C. In some embodiments, the first coupling agent is oxalyl chloride, the first catalyst N,N-dimethylformamide, the second base is an amine, the third solvent is an organic halogenated solvent, and the temperature is about −10° C. In some embodiments, the first coupling agent is oxalyl chloride, the first catalyst is N,N-dimethylformamide, the second base is triethylamine, the third solvent is an organic halogenated solvent, and the temperature is about −20 to about 10° C. In some embodiments, the first coupling agent is oxalyl chloride, the first catalyst N,N-dimethylformamide, the second base is triethylamine, the third solvent is an organic halogenated solvent, and the temperature is about −10° C. In some embodiments, the first coupling agent is oxalyl chloride, the first catalyst is N,N-dimethylformamide, the second base is triethylamine, the third solvent is dichloromethane, and the temperature is about −20 to about 10° C. In some embodiments, the first coupling agent is oxalyl chloride, the first catalyst is N,N-dimethylformamide, the second base is triethylamine, the third solvent is dichloromethane, and the temperature is about −10° C.

In some embodiments, the compound of Formula E is a salt, comprising a coformer.

In some embodiments, the coformer is an inorganic acid or an organic acid. In some embodiments, the coformer is a carboxylic acid or a sulfonic acid. In some embodiments, the coformer is sulfuric acid, hydrochloric acid, hydrobromic acid, acetic acid, pivalic acid, trifluoroacetic acid, phosphoric acid, methanesulfonic acid, p-toluenesulfonic acid, mandelic acid, tartaric acid, citric acid, benzenesulfonic acid, vanillic acid, formic acid, oxalic acid, fumaric acid, maleic acid, malic acid, succinic acid, cinnamic acid, or a combination thereof. In some embodiments, the coformer is sulfuric acid, hydrochloric acid, hydrobromic acid, acetic acid, pivalic acid, trifluoroacetic acid, phosphoric acid, methanesulfonic acid, p-toluenesulfonic acid, or a combination thereof. In some embodiments, the coformer is an inorganic acid. In some embodiments, the coformer is sulfuric acid, hydrochloric acid, hydrobromic acid, phosphoric acid, or a combination thereof. In some embodiments, the coformer is an organic acid where, in some embodiments, the organic acid is a carboxylic acid, a sulfonic acid, or a combination thereof. In some embodiments, the coformer is a carboxylic acid. In some embodiments, the coformer is acetic acid, pivalic acid, trifluoroacetic acid, mandelic acid, tartaric acid, citric acid, vanillic acid, formic acid, oxalic acid, fumaric acid, maleic acid, malic acid, succinic acid, cinnamic acid, or a combination thereof. In some embodiments, the coformer is a sulfonic acid. In some embodiments, the coformer is methanesulfonic acid, p-toluenesulfonic acid, benzenesulfonic acid or a combination thereof.

In some embodiments, the method further comprises preparing the compound of Formula A by treating a compound of Formula F:

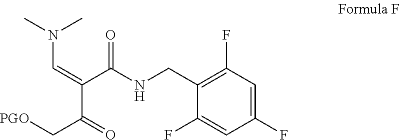

wherein PG is a hydroxyl protecting group as described herein, with a third base and a dicarbonyl reagent.

In some embodiments, the third base is a carbonate, a metal hydride, an alkoxide, a hydroxide, an amide, an organolithium reagent, an amine, a basic aromatic compound, or a combination thereof. In some embodiments, the third base is lithium carbonate, sodium carbonate, potassium carbonate, cesium carbonate, lithium hydride, sodium hydride, potassium hydride, sodium methoxide, sodium ethoxide, sodium tert-butoxide, sodium tert-pentoxide, lithium tert-butoxide, potassium ethoxide, potassium tert-butoxide, potassium tert-pentoxide, sodium hydroxide, potassium hydroxide, lithium bis(trimethylsilyl)amide, potassium bis(trimethylsilyl)amide, lithium diisopropylamide, n-butyllithium, phenyllithium, triethylamine, diisopropylethylamine, 1,8-diazabicyclo[5.4.0]undec-7-ene, pyridine, 2,6-lutidine, imidazole, or a combination thereof. In some embodiments, the third base is a carbonate. In some embodiments, the third base is lithium carbonate, sodium carbonate, potassium carbonate, cesium carbonate, or a combination thereof. In some embodiments, the third base is a metal hydride. In some embodiments, the third base is lithium hydride, sodium hydride, potassium hydride, or a combination thereof. In some embodiments, the third base is an alkoxide. In some embodiments, the third base is sodium methoxide, sodium ethoxide, sodium tert-butoxide, sodium tert-pentoxide, lithium tert-butoxide, potassium ethoxide, potassium tert-butoxide, potassium tert-pentoxide, or a combination thereof. In some embodiments, the third base is a hydroxide. In some embodiments, the third base is sodium hydroxide, potassium hydroxide, or a combination thereof. In some embodiments, the third base is an amide. In some embodiments, the third base is lithium bis(trimethylsilyl)amide, potassium bis(trimethylsilyl)amide, lithium diisopropylamide, or a combination thereof. In some embodiments, the third base is an organolithium reagent. In some embodiments, the third base is n-butyllithium, phenyllithium, or a combination thereof. In some embodiments, the third base is an amine. In some embodiments, the third base is triethylamine, diisopropylethylamine, 1,8-diazabicyclo[5.4.0]undec-7-ene, or a combination thereof. In some embodiments, the third base is a basic aromatic compound. In some embodiments, the third base is pyridine, 2,6-lutidine, imidazole, or a combination thereof. In some embodiments, the third base is sodium methoxide or sodium ethoxide. In some embodiments, the third base is sodium methoxide. In some embodiments, the third base is sodium ethoxide.

In some embodiments, the dicarbonyl reagent is dimethyl oxalate, diethyl oxalate, oxalyl chloride, a chlorooxoacetate, or a combination thereof. In some embodiments, the dicarbonyl reagent is dimethyl oxalate, diethyl oxalate, oxalyl chloride, or a combination thereof. In some embodiments, the dicarbonyl reagent is a chlorooxoacetate. In some embodiments, the dicarbonyl reagent is methyl chlorooxoacetate, ethyl chlorooxoacetate, or a combination thereof. In some embodiments, the dicarbonyl reagent is dimethyl oxalate, diethyl oxalate, oxalyl chloride, methyl chlorooxoacetate, ethyl chlorooxoacetate, or a combination thereof. In some embodiments, the dicarbonyl reagent is dimethyl oxalate or diethyl oxalate. In some embodiments, the dicarbonyl reagent is dimethyl oxalate. In some embodiments, the dicarbonyl reagent is diethyl oxalate.

In some embodiments, the compound of Formula F is further treated with a first promoter.

In some embodiments, the first promoter is a carboxylic acid, a sulfonic acid, a halogenated alcohol, a Lewis acid, an inorganic acid, or a combination thereof. In some embodiments, the first promoter is a carboxylic acid. In some embodiments, the first promoter is formic acid, acetic acid, trifluoroacetic acid, trichloroacetic acid, or a combination thereof. In some embodiments, the first promoter is a sulfonic acid. In some embodiments, the first promoter is methanesulfonic acid, p-toluenesulfonic acid, or a combination thereof. In some embodiments, the first promoter is a halogenated alcohol. In some embodiments, the first promoter is 2,2,2-trifluoroethanol, 2,2,2-trichloroethanol, 1,1,1,3,3,3-hexafluoro-2-propanol, or a combination thereof. In some embodiments, the first promoter is a Lewis acid. In some embodiments, the first promoter is boron trifluoride methyl etherate, boron trifluoride dibutyl etherate, boron trichloride, boron tribromide, aluminum chloride, aluminum bromide, or a combination thereof. In some embodiments, the first promoter is an inorganic acid. In some embodiments, the first promoter is hydrochloric acid, phosphoric acid, sulfuric acid, or a combination thereof. In some embodiments, the first promoter is formic acid, acetic acid, trifluoroacetic acid, trichloroacetic acid, methanesulfonic acid, p-toluenesulfonic acid, 2,2,2-trifluoroethanol, 2,2,2-trichloroethanol, 1,1,1,3,3,3-hexafluoro-2-propanol, boron trifluoride methyl etherate, boron trifluoride dibutyl etherate, boron trichloride, boron tribromide, aluminum chloride, aluminum bromide, hydrochloric acid, phosphoric acid, sulfuric acid, or a combination thereof. In some embodiments, the first promoter is 2,2,2-trifluoroethanol.

In some embodiments, the dicarbonyl reagent is dimethyl oxalate and the first promoter is 2,2,2-trifluoroethanol.

In some embodiments, the dicarbonyl reagent is diethyl oxalate and the first promoter is 2,2,2-trifluoroethanol.

In some embodiments, the compound of Formula F is treated with a third base and a dicarbonyl reagent, and optionally with a first promoter in a fourth solvent.

Any suitable solvent could be used as the fourth solvent. In some embodiments, the fourth solvent is an alcohol, an ester, an ether, a halogenated solvent, an aliphatic hydrocarbon, an aromatic hydrocarbon, a nitrile, a polar aprotic solvent, or a combination thereof. In some embodiments, the fourth solvent is methanol, ethanol, 2-propanol, ethyl acetate, isopropyl acetate, tetrahydrofuran, 2-methyltetrahydrofuran, tert-butyl methyl ether, dichloromethane, 1,2-dichloroethane, chlorobenzene, benzene, toluene, n-heptane, acetonitrile, N,N-dimethylformamide, N,N-dimethylacetamide, N-methyl-2-pyrrolidone, or a combination thereof. In some embodiments, the fourth solvent is methanol, N,N-dimethylacetamide, or a combination thereof. In some embodiments, the fourth solvent is a combination of N,N-dimethylacetamide and methanol. In some embodiments, the fourth solvent is an organic alcohol. In some embodiments, the fourth solvent is methanol, ethanol, 2-propanol, or a combination thereof. In some embodiments, the fourth solvent is an organic ester. In some embodiments, the fourth solvent is ethyl acetate, isopropyl acetate, or a combination thereof. In some embodiments, the fourth solvent is an organic ether. In some embodiments, the fourth solvent is tetrahydrofuran, 2-methyltetrahydrofuran, tert-butyl methyl ether, or a combination thereof. In some embodiments, the fourth solvent is an organic halogenated solvent. In some embodiments, the fourth solvent is dichloromethane, 1,2-dichloroethane, chlorobenzene, or a combination thereof. In some embodiments, the fourth solvent is an organic aliphatic hydrocarbon. In some embodiments, the fourth solvent is heptane (e.g., n-heptane). In some embodiments, the fourth solvent is an organic aromatic hydrocarbon. In some embodiments, the fourth solvent is benzene, toluene, or a combination thereof. In some embodiments, the fourth solvent is an organic nitrile. In some embodiments, the fourth solvent is acetonitrile. In some embodiments, the fourth solvent is a polar aprotic solvent. In some embodiments, the fourth solvent is N,N-dimethylformamide, N,N-dimethylacetamide, N-methyl-2-pyrrolidone, or a combination thereof. In some embodiments, the fourth solvent is methanol, ethanol, N,N-dimethylacetamide, or a combination thereof. In some embodiments, the fourth solvent is a combination of N,N-dimethylacetamide and methanol. In some embodiments, the fourth solvent is a combination of N,N-dimethylacetamide and ethanol.

In some embodiments, the compound of Formula F is treated with the third base and the dicarbonyl reagent, and optionally a first promoter, in a fourth solvent at a temperature of about −30 to about 60° C. In some embodiments, the compound of Formula F is treated with the third base and the dicarbonyl reagent, and optionally a first promoter, in a fourth solvent at a temperature of about 0 to about 20° C. In some embodiments, the compound of Formula F is treated with the third base and the dicarbonyl reagent, and optionally a first promoter, in a fourth solvent at a temperature of about 10° C.

In some embodiments, the third base is sodium methoxide, the dicarbonyl reagent is dimethyl oxalate, the first promoter is 2,2,2-trifluoroethanol, the fourth solvent is a combination of N,N-dimethylacetamide and methanol, and the temperature is about 0 to about 20° C. In some embodiments, the third base is sodium methoxide, the dicarbonyl reagent is dimethyl oxalate, the first promoter is 2,2,2-trifluoroethanol, the fourth solvent is a combination of N,N-dimethylacetamide and methanol, and the temperature is about 10° C.

In some embodiments, the third base is sodium ethoxide, the dicarbonyl reagent is diethyl oxalate, the first promoter is 2,2,2-trifluoroethanol, the fourth solvent is a combination of N,N-dimethylacetamide and ethanol, and the temperature is about 0 to about 20° C. In some embodiments, the third base is sodium ethoxide, the dicarbonyl reagent is diethyl oxalate, the first promoter is 2,2,2-trifluoroethanol, the fourth solvent is a combination of N,N-dimethylacetamide and ethanol, and the temperature is about 10° C.

In some embodiments, the method further comprises preparing the compound of Formula F by treating a compound of Formula G:

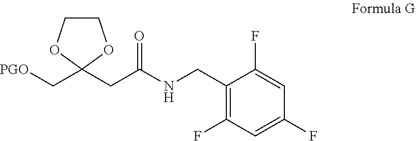

with a first acid.

In some embodiments, the first acid is formic acid, hydrochloric acid, hydrobromic acid, sulfuric acid, a sulfonic acid, acetic acid, trifluoroacetic acid, phosphoric acid, oxalic acid, or a combination thereof. In some embodiments, the first acid is formic acid, hydrochloric acid, hydrobromic acid, sulfuric acid, methanesulfonic acid, p-toluenesulfonic acid, benzenesulfonic acid, acetic acid, trifluoroacetic acid, phosphoric acid, oxalic acid, or a combination thereof. In some embodiments, the first acid is a sulfonic acid. In some embodiments, the first acid is methanesulfonic acid, p-toluenesulfonic acid, benzenesulfonic acid, or a combination thereof. In some embodiments, the first acid is formic acid. In some embodiments, the first acid further comprises water.

In some embodiments, the compound of Formula G is treated with the first acid in absence of any solvent.

In some embodiments, the compound of Formula G is treated with the first acid in the presence of a fifth solvent.

In some embodiments, the fifth solvent is an alcohol, an ether, a halogenated solvent, an aliphatic hydrocarbon solvent, an aromatic hydrocarbon solvent, an organic nitrile solvent, a polar aprotic solvent, or combination thereof. In some embodiments, the fifth solvent is methanol, ethanol, 2-propanol, tetrahydrofuran, 2-methyltetrahydrofuran, tert-butyl methyl ether, dichloromethane, 1,2-dichloroethane, chlorobenzene, toluene, n-heptane, acetonitrile, N,N-dimethylformamide, N,N-dimethylacetamide, N-methyl-2-pyrrolidone, or a combination thereof. In some embodiments, the fifth solvent is an organic alcohol. In some embodiments, the fifth solvent is methanol, ethanol, 2-propanol, or combination thereof. In some embodiments, the fifth solvent is an organic ether. In some embodiments, the fifth solvent is tetrahydrofuran, 2-methyltetrahydrofuran, tert-butyl methyl ether, or combination thereof. In some embodiments, the fifth solvent is a halogenated solvent. In some embodiments, the fifth solvent is dichloromethane, 1,2-dichloroethane, chlorobenzene, or combination thereof. In some embodiments, the fifth solvent is an aliphatic hydrocarbon solvent. In some embodiments, the fifth solvent is heptane (e.g., n-heptane). In some embodiments, the fifth solvent is an aromatic hydrocarbon solvent. In some embodiments, the fifth solvent is toluene. In some embodiments, the fifth solvent is an organic nitrile solvent. In some embodiments, the fifth solvent is acetonitrile. In some embodiments, the fifth solvent is a polar aprotic solvent. In some embodiments, the fifth solvent is N,N-dimethylformamide, N,N-dimethylacetamide, N-methyl-2-pyrrolidone, or combination thereof.

In some embodiments, the compound of Formula G is treated with a first acid further in the presence of water.

In some embodiments, the compound of Formula G is treated with the first acid at a temperature of about 20 to about 100° C. In some embodiments, the compound of Formula G is treated with the first acid at a temperature of about 45 to about 85° C. In some embodiments, the compound of Formula G is treated with the first acid at a temperature of about 65° C.

In some embodiments, the first acid is formic acid, the fifth solvent is absent, and the temperature is about 45 to about 85° C. In some embodiments, the first acid is formic acid, the fifth solvent is absent, and the temperature is about 65° C.

In some embodiments, treatment of the compound of Formula G with the first acid provides a compound of Formula H:

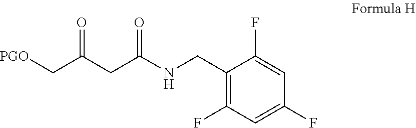

and wherein the compound of Formula H is treated with an electrophile and a second acid to provide the compound of Formula F.

In some embodiments, the electrophile is N,N-diethylformamide or a formamide acetal. In some embodiments, the electrophile is N,N-diethylformamide. In some embodiments, the electrophile is a formamide acetal. In some embodiments, the electrophile is N,N-dimethylformamide diisopropyl acetal, N,N-dimethylformamide dimethylacetal, N,N-dimethylformamide diethylacetal, or a combination thereof. In some embodiments, the electrophile is N,N-dimethylformamide dimethylacetal.

In some embodiments, the second acid is an inorganic acid, an organic acid, a metal chloride or a metal bromide. In some embodiments, the second acid is an inorganic acid. In some embodiments, the second acid is sulfuric acid, nitric acid, hydrobromic acid, phosphoric acid, or a combination thereof. In some embodiments, the second acid is an organic acid. In some embodiments, the second acid is methanesulfonic acid, trifluoroacetic acid, camphorsulfonic acid, p-toluenesulfonic acid, triflic acid, acetic acid, pivalic acid, or a combination thereof. In some embodiments, the second acid is a metal chloride. In some embodiments, the second acid is lithium chloride, magnesium chloride, aluminum chloride, or a combination thereof. In some embodiments, the second acid is a metal bromide. In some embodiments, the second acid is lithium bromide, magnesium bromide, aluminum bromide, or a combination thereof. In some embodiments, the second acid is sulfuric acid, nitric acid, hydrobromic acid, phosphoric acid, methanesulfonic acid, trifluoroacetic acid, camphorsulfonic acid, p-toluenesulfonic acid, triflic acid, acetic acid, pivalic acid, lithium chloride, magnesium chloride, aluminum chloride, lithium bromide, magnesium bromide, aluminum bromide, or a combination thereof. In some embodiments, the second acid is trifluoroacetic acid.

In some embodiments, the electrophile is a formamide acetal and the second acid is an inorganic acid. In some embodiments, the electrophile is N,N-dimethylformamide dimethylacetal and the second acid is an inorganic acid. In some embodiments, the electrophile is N,N-dimethylformamide dimethylacetal and the second acid is trifluoroacetic acid.

In some embodiments, the compound of Formula H is treated with the electrophile and the second acid further in the presence of a fourth base.

In some embodiments, the fourth base is a carbonate, a bicarbonate, a hydroxide, an amine, a basic aromatic compound, an amide, or a combination thereof. In some embodiments, the fourth base is a carbonate. In some embodiments, the fourth base is lithium carbonate, sodium carbonate, potassium carbonate, cesium carbonate, or a combination thereof. In some embodiments, the fourth base is a bicarbonate. In some embodiments, the fourth base is lithium bicarbonate, sodium bicarbonate, potassium bicarbonate, or a combination thereof. In some embodiments, the fourth base is a hydroxide. In some embodiments, the fourth base is sodium hydroxide, potassium hydroxide, or a combination thereof. In some embodiments, the fourth base is an amine. In some embodiments, the fourth base is triethylamine, diisopropylethylamine, or a combination thereof. In some embodiments, the fourth base is a basic aromatic compound. In some embodiments, the fourth base is pyridine, 2,6-lutidine, imidazole, or a combination thereof. In some embodiments, the fourth base is an amide. In some embodiments, the fourth base is lithium bis(trimethylsilyl)amide, potassium bis(trimethylsilyl)amide, lithium diisopropylamide, or a combination thereof. In some embodiments, the fourth base is lithium carbonate, sodium carbonate, potassium carbonate, cesium carbonate, lithium bicarbonate, sodium bicarbonate, potassium bicarbonate, sodium hydroxide, potassium hydroxide, triethylamine, diisopropylethylamine, pyridine, 2,6-lutidine, imidazole, lithium bis(trimethylsilyl)amide, potassium bis(trimethylsilyl)amide, lithium diisopropylamide, or a combination thereof.

In some embodiments, the compound of Formula H is treated with an electrophile and a second acid further in the absence of any base.

In some embodiments, the compound of Formula H is treated with the electrophile and the second acid, optionally in presence of the fourth base, further in presence of a sixth solvent. Any suitable solvent could be used as the sixth solvent.

In some embodiments, the sixth solvent is an ether, a halogenated solvent, an aliphatic hydrocarbon, an aromatic hydrocarbon, a nitrile, a polar aprotic solvent, or a combination thereof. In some embodiments, the sixth solvent is an ether. In some embodiments, the sixth solvent is tetrahydrofuran, 2-methyltetrahydrofuran, tert-butyl methyl ether, or a combination thereof. In some embodiments, the sixth solvent is a halogenated solvent. In some embodiments, the sixth solvent is dichloromethane, 1,2-dichloroethane, chlorobenzene, or a combination thereof. In some embodiments, the sixth solvent is an aliphatic hydrocarbon. In some embodiments, the sixth solvent is heptane (e.g. n-heptane). In some embodiments, the sixth solvent is an aromatic hydrocarbon. In some embodiments, the sixth solvent is toluene. In some embodiments, the sixth solvent is a nitrile. In some embodiments, the sixth solvent is acetonitrile. In some embodiments, the sixth solvent is a polar aprotic solvent. In some embodiments, the sixth solvent is N,N-dimethylformamide, N,N-dimethylacetamide, N-methyl-2-pyrrolidone, or a combination thereof. In some embodiments, the sixth solvent is tetrahydrofuran, 2-methyltetrahydrofuran, tert-butyl methyl ether, dichloromethane, 1,2-dichloroethane, chlorobenzene, n-heptane, toluene, acetonitrile, N,N-dimethylformamide, N,N-dimethylacetamide, N-methyl-2-pyrrolidone, or a combination thereof. In some embodiments, the sixth solvent is toluene.

In some embodiments, the electrophile is a formamide acetal, the second acid is an inorganic acid, and the sixth solvent is an aromatic hydrocarbon. In some embodiments, the electrophile is N,N-dimethylformamide dimethylacetal, the second acid is an inorganic acid, and the sixth solvent is an aromatic hydrocarbon. In some embodiments, the electrophile is N,N-dimethylformamide dimethylacetal, the second acid is trifluoroacetic acid, and the sixth solvent is an aromatic hydrocarbon.

In some embodiments, the electrophile is a formamide acetal, the second acid is an inorganic acid, and the sixth solvent is toluene. In some embodiments, the electrophile is N,N-dimethylformamide dimethylacetal, the second acid is an inorganic acid, and the sixth solvent is toluene. In some embodiments, the electrophile is N,N-dimethylformamide dimethylacetal, the second acid is trifluoroacetic acid, and the sixth solvent is toluene.

In some embodiments, the electrophile is a formamide acetal, the second acid is an inorganic acid, the sixth solvent is an aromatic hydrocarbon, and the fourth base is absent. In some embodiments, the electrophile is N,N-dimethylformamide dimethylacetal, the second acid is an inorganic acid, the sixth solvent is an aromatic hydrocarbon, and the fourth base is absent. In some embodiments, the electrophile is N,N-dimethylformamide dimethylacetal, the second acid is trifluoroacetic acid, the sixth solvent is an aromatic hydrocarbon, and the fourth base is absent.

In some embodiments, the electrophile is a formamide acetal, the second acid is an inorganic acid, the sixth solvent is toluene, and the fourth base is absent. In some embodiments, the electrophile is N,N-dimethylformamide dimethylacetal, the second acid is an inorganic acid, the sixth solvent is toluene, and the fourth base is absent. In some embodiments, the electrophile is N,N-dimethylformamide dimethylacetal, the second acid is trifluoroacetic acid, the sixth solvent is toluene, and the fourth base is absent.

In some embodiments, the compound of Formula H is treated with the electrophile and the second acid, optionally in presence of the fourth base, in the sixth solvent at a temperature of about 20 to about 130° C. In some embodiments, the compound of Formula His treated with the electrophile and the second acid, optionally in presence of the fourth base, in the sixth solvent at a temperature of about 80 to about 100° C. In some embodiments, the compound of Formula H is treated with the electrophile and the second acid, optionally in presence of the fourth base, in the sixth solvent at a temperature of about 90° C.

In some embodiments, the electrophile is N,N-dimethylformamide dimethylacetal, the second acid is trifluoroacetic acid, and the temperature is about 80 to about 100° C. In some embodiments, the electrophile is N,N-dimethylformamide dimethylacetal, the second acid is trifluoroacetic acid, and the temperature is about 90° C.

In some embodiments, the electrophile is N,N-dimethylformamide dimethylacetal, the second acid is trifluoroacetic acid, the fourth base is absent, and the temperature is about 80 to about 100° C. In some embodiments, the electrophile is N,N-dimethylformamide dimethylacetal, the second acid is trifluoroacetic acid, the fourth base is absent, and the temperature is about 90° C.

In some embodiments, the electrophile is N,N-dimethylformamide dimethylacetal, the second acid is trifluoroacetic acid, the fourth base is absent, the sixth solvent is toluene, and the temperature is about 80 to about 100° C. In some embodiments, the electrophile is N,N-dimethylformamide dimethylacetal, the second acid is trifluoroacetic acid, the fourth base is absent, the sixth solvent is toluene, and the temperature is about 90° C.

In some embodiments, the method further comprises preparing the compound of Formula G by treating a compound of Formula J:

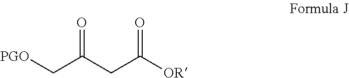

with a compound of Formula K:

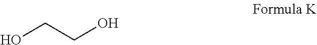

in the presence of a third acid and a second promoter, wherein R′ is C₁₋₆ alkyl, C₆₋₁₀ aryl-C₁₋₄ alkylene, or C₆₋₁₀ aryl.

In some embodiments, the third acid is a carboxylic acid, a sulfonic acid, an inorganic acid, or a combination thereof. In some embodiments, the third acid is a carboxylic acid. In some embodiments, the third acid is acetic acid. In some embodiments, the third acid is a sulfonic acid. In some embodiments, the third acid is methanesulfonic acid, p-toluenesulfonic acid, benzenesulfonic acid, or a combination thereof. In some embodiments, the third acid is an inorganic acid. In some embodiments, the third acid is hydrochloric acid, sulfuric acid, phosphoric acid, or a combination thereof. In some embodiments, the third acid is acetic acid, methanesulfonic acid, p-toluenesulfonic acid, benzenesulfonic acid, hydrochloric acid, sulfuric acid, phosphoric acid, or a combination thereof. In some embodiments, the third acid is p-toluenesulfonic acid.

In some embodiments, the second promoter is trimethyl orthoformate, triethyl orthoformate, an orthoacetate, molecular sieves, magnesium sulfate, sodium sulfate, azeotropic distillation, or a combination thereof. In some embodiments, the second promoter is an orthoformate. In some embodiments, the second promoter is trimethyl orthoformate, triethyl orthoformate, or a combination thereof. In some embodiments, the second promoter is an orthoacetate. In some embodiments, the second promoter is trimethyl orthoacetate, triethyl orthoacetate, or a combination thereof. In some embodiments, the second promoter is a sulfate. In some embodiments, the second promoter is magnesium sulfate, sodium sulfate, or a combination thereof. In some embodiments, the second promoter is azeotropic distillation. In some embodiments, the second promoter is trimethyl orthoformate, triethyl orthoformate, trimethyl orthoacetate, triethyl orthoacetate, molecular sieves, magnesium sulfate, sodium sulfate, azeotropic distillation, or a combination thereof. In some embodiments, the second promoter is trimethyl orthoformate.

In some embodiments, the third acid is a sulfonic acid and the second promoter is an orthoformate. In some embodiments, the third acid is methanesulfonic acid, p-toluenesulfonic acid, benzenesulfonic acid, or a combination thereof and the second promoter is an orthoformate. In some embodiments, the third acid is p-toluenesulfonic acid and the second promoter is an orthoformate. In some embodiments, the third acid is a sulfonic acid and the second promoter is trimethyl orthoformate, triethyl orthoformate, or a combination thereof. In some embodiments, the third acid is methanesulfonic acid, p-toluenesulfonic acid, benzenesulfonic acid, or a combination thereof and the second promoter is trimethyl orthoformate, triethyl orthoformate, or a combination thereof. In some embodiments, the third acid is p-toluenesulfonic acid and the second promoter is trimethyl orthoformate, triethyl orthoformate, or a combination thereof. In some embodiments, the third acid is a sulfonic acid and the second promoter is trimethyl orthoformate. In some embodiments, the third acid is methanesulfonic acid, p-toluenesulfonic acid, benzenesulfonic acid, or a combination thereof and the second promoter is trimethyl orthoformate. In some embodiments, the third acid is p-toluenesulfonic acid and the second promoter is trimethyl orthoformate.

In some embodiments, the compound of Formula J is treated with the compound of Formula K in the presence of the third acid and the second promoter further in the presence of a seventh solvent. Any suitable solvent can be used as the seventh solvent.

In some embodiments, the seventh solvent is an ester, an ether, an aliphatic hydrocarbon, an aromatic hydrocarbon, a nitrile, polar aprotic solvent, or a combination thereof. In some embodiments, the seventh solvent is an ester. In some embodiments, the seventh solvent is ethyl acetate, isopropyl acetate, or a combination thereof. In some embodiments, the seventh solvent is an ether. In some embodiments, the seventh solvent is tetrahydrofuran, 2-methyltetrahydrofuran, tert-butyl methyl ether, or a combination thereof. In some embodiments, the seventh solvent is an aliphatic hydrocarbon. In some embodiments, the seventh solvent is heptane (e.g., n-heptane). In some embodiments, the seventh solvent is an aromatic hydrocarbon. In some embodiments, the seventh solvent is toluene. In some embodiments, the seventh solvent is a nitrile. In some embodiments, the seventh solvent is acetonitrile. In some embodiments, the seventh solvent is a polar aprotic solvent. In some embodiments, the seventh solvent is N,N-dimethylformamide, N,N-dimethylacetamide, N-methyl-2-pyrrolidone, or a combination thereof. In some embodiments, the seventh solvent is ethyl acetate, isopropyl acetate, tetrahydrofuran, 2-methyltetrahydrofuran, tert-butyl methyl ether, toluene, n-heptane, acetonitrile, N,N-dimethylformamide, N,N-dimethylacetamide, N-methyl-2-pyrrolidone, or a combination thereof.

In some embodiments, the compound of Formula J is treated with the compound of Formula K in the presence of the third acid and the second promoter further in the absence of any solvent, i.e. the seventh solvent is absent.

In some embodiments, the compound of Formula J is treated with the compound of Formula K in the presence of the third acid and the second promoter, optionally in presence of a seventh solvent, at a temperature of about 10 to about 70° C. In some embodiments, the compound of Formula J is treated with the compound of Formula K in the presence of the third acid and the second promoter, optionally in presence of a seventh solvent, at a temperature of about 30 to about 50° C. In some embodiments, the compound of Formula J is treated with the compound of Formula K in the presence of the third acid and the second promoter, optionally in presence of a seventh solvent, at a temperature of about 40° C.

In some embodiments, the third acid a sulfonic acid, the second promoter is an orthoformate, and the temperature is about 30 to about 50° C. In some embodiments, the third acid is a sulfonic acid, the second promoter is an orthoformate, and the temperature is about 40° C.

In some embodiments, the third acid p-toluenesulfonic acid, the second promoter is an orthoformate, and the temperature is about 30 to about 50° C. In some embodiments, the third acid is p-toluenesulfonic acid, the second promoter is an orthoformate, and the temperature is about 40° C.

In some embodiments, the third acid a sulfonic acid, the second promoter is trimethyl orthoformate, and the temperature is about 30 to about 50° C. In some embodiments, the third acid is a sulfonic acid, the second promoter is trimethyl orthoformate, and the temperature is about 40° C.

In some embodiments, the third acid is p-toluenesulfonic acid, the second promoter is trimethyl orthoformate, and the temperature is about 30 to about 50° C. In some embodiments, the third acid is p-toluenesulfonic acid, the second promoter is trimethyl orthoformate, and the temperature is about 40° C.

In some embodiments, the third acid is p-toluenesulfonic acid, the second promoter is trimethyl orthoformate, the seventh solvent is absent, and the temperature is about 30 to about 50° C. In some embodiments, the third acid is p-toluenesulfonic acid, the second promoter is trimethyl orthoformate, the seventh solvent is absent, and the temperature is about 40° C.

In some embodiments, treatment of the compound of Formula J with the compound of Formula K provides a compound of Formula L:

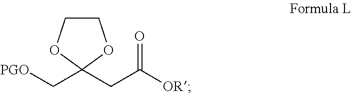

and wherein the method further comprises treating the compound of Formula L with a fifth base.

In some embodiments, the fifth base is a hydroxide, a carbonate, a phosphate, or a combination thereof. In some embodiments, the fifth base is a hydroxide. In some embodiments, the fifth base is sodium hydroxide, lithium hydroxide, potassium hydroxide, ammonium hydroxide, or a combination thereof. In some embodiments, the fifth base is a carbonate. In some embodiments, the fifth base is lithium carbonate, sodium carbonate, cesium carbonate, or a combination thereof. In some embodiments, the fifth base is a phosphate. In some embodiments, the fifth base is potassium phosphate dibasic, potassium phosphate tribasic, sodium phosphate dibasic, sodium phosphate tribasic, or a combination thereof. In some embodiments, the fifth base is sodium hydroxide, lithium hydroxide, potassium hydroxide, ammonium hydroxide, lithium carbonate, sodium carbonate, cesium carbonate, potassium phosphate dibasic, potassium phosphate tribasic, sodium phosphate dibasic, sodium phosphate tribasic, or a combination thereof. In some embodiments, the fifth base is sodium hydroxide.

In some embodiments, the compound of Formula L is treated with the fifth base further in the presence of an eighth solvent.

In some embodiments, the eighth solvent is an ether, a ketone, a nitrile, a polar aprotic solvent, water, or a combination thereof. In some embodiments, the eighth solvent is an ether. In some embodiments, the eighth solvent is tetrahydrofuran, 2-methyltetrahydrofuran, tert-butyl methyl ether, or a combination thereof. In some embodiments, the eighth solvent is a combination of ether and water. In some embodiments, the eighth solvent is water, tetrahydrofuran, 2-methyltetrahydrofuran, tert-butyl methyl ether, or a combination thereof. In some embodiments, the eighth solvent is a ketone. In some embodiments, the eighth solvent is a combination of a ketone and water. In some embodiments, the eighth solvent is a nitrile solvent. In some embodiments, the eighth solvent is a combination of a nitrile solvent and water. In some embodiments, the eighth solvent is a polar aprotic solvent. In some embodiments, the eighth solvent is a combination of a polar aprotic solvent and water. In some embodiments, the eighth solvent is tetrahydrofuran, 2-methyltetrahydrofuran, tert-butyl methyl ether, acetone, 2-butanone, acetonitrile, N,N-dimethylformamide, N,N-dimethylacetamide, N-methyl-2-pyrrolidone, water, or a combination thereof. In some embodiments, the eighth solvent is water.

In some embodiments, the fifth base is a hydroxide and the eighth solvent is water. In some embodiments, the fifth base is sodium hydroxide, lithium hydroxide, potassium hydroxide, ammonium hydroxide, or a combination thereof and the eighth solvent is water. In some embodiments, the fifth base is sodium hydroxide and the eighth solvent is water.

In some embodiments, the compound of Formula L is treated with the fifth base further in the presence of a second catalyst.

Any suitable catalyst could be used as the second catalyst. In some embodiments, the second catalyst is a phase transfer catalyst. In some embodiments, the second catalyst is an ammonium salt, a phosphonium salt, hydrogen peroxide, or a combination thereof. In some embodiments, the second catalyst is an ammonium salt. In some embodiments, the second catalyst is tetrabutylammonium chloride, tetrabutylammonium hydroxide, benzyltributylammonium bromide, 1-methylimidazolium hydrogen sulfate, or a combination thereof. In some embodiments, the second catalyst is a phosphonium salt. In some embodiments, the second catalyst is tetrabutylphosphonium chloride. In some embodiments, the second catalyst is hydrogen peroxide. In some embodiments, the second catalyst is tetrabutylammonium chloride, tetrabutylammonium hydroxide, benzyltributylammonium bromide, 1-methylimidazolium hydrogen sulfate, tetrabutylphosphonium chloride, hydrogen peroxide, or a combination thereof.

In some embodiments, the compound of Formula L is treated with the fifth base in absence of a second catalyst.

In some embodiments, the compound of Formula L is treated with the fifth base at a temperature of about 5 to about 60° C. In some embodiments, the compound of Formula L is treated with the fifth base at a temperature of about 10 to about 30° C. In some embodiments, the compound of Formula L is treated with the fifth base at a temperature of about 20° C.

In some embodiments, the fifth base is a hydroxide, the eighth solvent is water, the second catalyst if present is a phase transfer catalyst, and the temperature is about 10 to about 30° C. In some embodiments, the fifth base is a hydroxide, the eighth solvent is water, the second catalyst if present is a phase transfer catalyst, and the temperature is about 20° C.

In some embodiments, the fifth base is sodium hydroxide, the eighth solvent is water, the second catalyst if present is a phase transfer catalyst, and the temperature is about 10 to about 30° C. In some embodiments, the fifth base is sodium hydroxide, the eighth solvent is water, the second catalyst is absent and the temperature is about 20° C.

In some embodiments, the fifth base is a hydroxide, the eighth solvent is water, the second catalyst if present is a phase transfer catalyst, and the temperature is about 10 to about 30° C. In some embodiments, the fifth base is a hydroxide, the eighth solvent is water, the second catalyst if present is a phase transfer catalyst, and the temperature is about 20° C.

In some embodiments, the fifth base is sodium hydroxide, the eighth solvent is water, the second catalyst is absent and the temperature is about 10 to about 30° C. In some embodiments, the fifth base is sodium hydroxide, the eighth solvent is water, the second catalyst is absent and the temperature is about 20° C.

In some embodiments, treatment of the compound of Formula L with the fifth base provides a compound of Formula M:

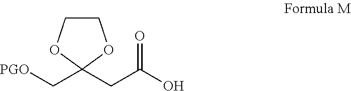

and wherein the method further comprises treating the compound of Formula M with a compound of Formula E:

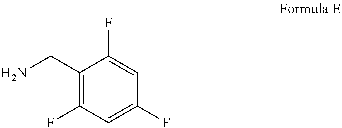 [SMILES: NCc1c(F)cc(F)cc1F]

or a salt thereof in the presence of a second coupling agent.

In some embodiments, the second coupling agent is oxalyl chloride, thionyl chloride, phosgene, triphosgene, an alkyl chloroformate, carbodiimide, propanephosphonic acid anhydride, a carboxylic anhydride, a carboxylic acid chloride, a peptide coupling agent, or a combination thereof. In some embodiments, the second coupling agent is oxalyl chloride, thionyl chloride, or a combination thereof. In some embodiments, the second coupling agent is phosgene, triphosgene, or a combination thereof. In some embodiments, the second coupling agent is an alkyl chloroformate. In some embodiments, the second coupling agent is ethyl chloroformate, isobutyl chloroformate, or a combination thereof. In some embodiments, the second coupling agent is carbodiimide. In some embodiments, the second coupling agent is dicyclohexylcarbodiimide, diisopropylcarbodiimide, 1-ethyl-3-(3-dimethylaminopropyl)carbodiimide), or a combination thereof. In some embodiments, the second coupling agent is propanephosphonic acid anhydride. In some embodiments, the second coupling agent is a carboxylic anhydride. In some embodiments, the second coupling agent is a carboxylic acid chloride. In some embodiments, the second coupling agent is a peptide coupling agent. In some embodiments, the second coupling agent is HATU, HBTU, TATU, TBTU, HCTU, BOP, PyBOP, COMU, or a combination thereof. In some embodiments, the second coupling agent is ethyl chloroformate, isobutyl chloroformate, dicyclohexylcarbodiimide, diisopropylcarbodiimide, 1-ethyl-3-(3-dimethylaminopropyl)carbodiimide), propanephosphonic acid anhydride, acetic anhydride, pivalic anhydride, benzoic anhydride, 2,4,6-trichlorobenzoyl chloride, carbonyl diimidazole, HATU, HBTU, TATU, TBTU, HCTU, BOP, PyBOP, COMU, or a combination thereof. In some embodiments, the second coupling agent is carbonyl diimidazole.

In some embodiments, the compound of Formula M is treated with the compound of Formula E or a salt thereof further in the presence of a ninth solvent.

In some embodiments, the ninth solvent is an ester, an ether, a halogenated solvent, an aliphatic hydrocarbon, an aromatic hydrocarbon, a ketone, a nitrile, a polar aprotic solvent, or a combination thereof. In some embodiments, the ninth solvent is an ester. In some embodiments, the ninth solvent is ethyl acetate, isopropyl acetate, or a combination thereof. In some embodiments, the ninth solvent is an ether. In some embodiments, the ninth solvent is tetrahydrofuran, 2-methyltetrahydrofuran, tert-butyl methyl ether, or a combination thereof. In some embodiments, the ninth solvent is a halogenated solvent. In some embodiments, the ninth solvent is dichloromethane, 1,2-dichloroethane, chlorobenzene, or a combination thereof. In some embodiments, the ninth solvent is an aliphatic hydrocarbon. In some embodiments, the ninth solvent is heptane (e.g., n-heptane). In some embodiments, the ninth solvent is an aromatic hydrocarbon. In some embodiments, the ninth solvent is toluene. In some embodiments, the ninth solvent is a ketone. In some embodiments, the ninth solvent is acetone, 2-butanone, or a combination thereof. In some embodiments, the ninth solvent is a nitrile. In some embodiments, the ninth solvent is acetonitrile. In some embodiments, the ninth solvent is a polar aprotic solvent. In some embodiments, the ninth solvent is N,N-dimethylformamide, N,N-dimethylacetamide, N-methyl-2-pyrrolidone, or a combination thereof. In some embodiments, the ninth solvent is ethyl acetate, isopropyl acetate, tetrahydrofuran, tert-butyl methyl ether, dichloromethane, 2-methyltetrahydrofuran, 1,2-dichloroethane, chlorobenzene, toluene, n-heptane, acetone, 2-butanone, acetonitrile, N,N-dimethylformamide, N,N-dimethylacetamide, N-methyl-2-pyrrolidone, or a combination thereof. In some embodiments, the ninth solvent is dichloromethane, 2-methyltetrahydrofuran, or a combination thereof. In some embodiments, the ninth solvent is dichloromethane. In some embodiments, the ninth solvent is 2-methyltetrahydrofuran. In some embodiments, the ninth solvent is a combination of dichloromethane and 2-methyltetrahydrofuran.

In some embodiments, the second coupling agent is carbonyl diimidazole and the ninth solvent comprises dichloromethane, 2-methyltetrahydrofuran, or a combination thereof.

In some embodiments, the compound of Formula M is treated with the compound of Formula E or a salt thereof at a temperature of about 0 to about 60° C. In some embodiments, the compound of Formula M is treated with the compound of Formula E or a salt thereof at a temperature of about 10 to about 30° C. In some embodiments, the compound of Formula M is treated with the compound of Formula E or a salt thereof at a temperature of about 20° C.

In some embodiments, the second coupling agent is carbonyl diimidazole, the ninth solvent is a combination of dichloromethane and 2-methyltetrahydrofuran, and the temperature is about 10 to about 30° C. In some embodiments, the second coupling agent is carbonyl diimidazole, the ninth solvent is a combination of dichloromethane and 2-methyltetrahydrofuran, and the temperature is about 20° C.

In the compounds, intermediates, and reactions disclosed herein, PG can be any suitable hydroxyl protecting group. In some embodiments, the hydroxyl is protected as an ether. In some embodiments, PG is benzyl or methyl. In some embodiments, PG is benzyl. In some embodiments, PG is methyl.

In the compounds, intermediates, and reactions disclosed herein, R is C₁₋₆ alkyl, C₆₋₁₀ aryl-C₁₋₄ alkylene, or C₆₋₁₀ aryl. In some embodiments, R is methyl, ethyl, tert-butyl, benzyl, or phenyl. In some embodiments, R is methyl, ethyl, or tert-butyl. In some embodiments, R is methyl or, ethyl. In some embodiments, R is benzyl. In some embodiments, R is phenyl. In some embodiments, R is methyl.

In some embodiments, R′ is C₁₋₆ alkyl, C₆₋₁₀ aryl-C₁₋₄ alkylene, or C₆₋₁₀ aryl. In some embodiments, R′ is methyl, ethyl, isopropyl, tert-butyl, benzyl, or phenyl. In some embodiments, R′ is methyl, ethyl, isopropyl, or tert-butyl. In some embodiments, R′ is benzyl. In some embodiments, R′ is phenyl. In some embodiments, R′ is ethyl.

In some embodiments, the compound of Formula B is

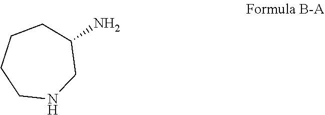

or a salt thereof. As such, the disclosure provides a method of preparing a compound of Formula I-A:

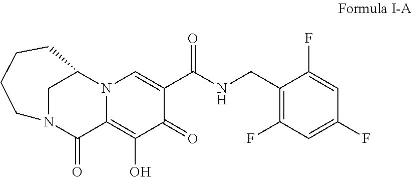 [SMILES: O=C(NCc1c(F)cc(F)cc1F)c1cn2c(c(O)c1=O)C(=O)N1CCCC[C@H]2C1]

or a salt thereof, wherein the method comprises treating a compound of Formula A:

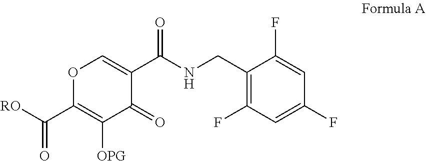

with a compound of Formula B-A:

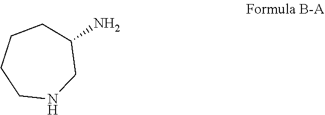

or a salt thereof, wherein PG is a hydroxyl protecting group and R is C₁₋₆ alkyl, C₆₋₁₀ aryl-C₁₋₄ alkylene, or C₆₋₁₀ aryl as described herein.

In some embodiments, the treatment of the compound of Formula A with a compound of Formula B-A or a salt thereof provides a compound of Formula C-A:

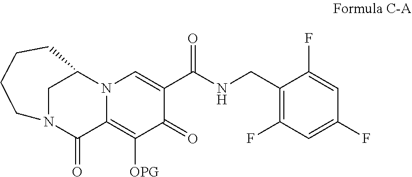

wherein deprotection of the compound of Formula C-A provides a compound of Formula I-A or a salt thereof.

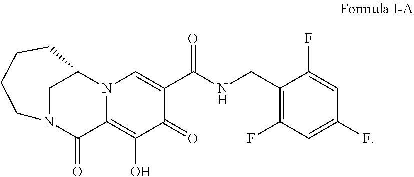 [SMILES: O=C(NCc1c(F)cc(F)cc1F)c1cn2c(c(O)c1=O)C(=O)N1CCCC[C@H]2C1]

In some embodiments, the method further comprises preparing a salt of a compound of Formula I-A, wherein the salt has the Formula I-A-X:

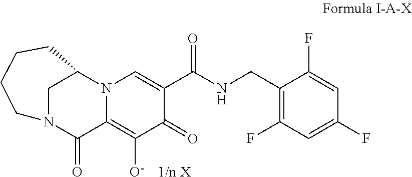

wherein n is 1 or 2 and X is selected from sodium (Na⁺), potassium (K⁺), and calcium (Ca²⁺).

In one embodiment, n is 1 and X is sodium (Na⁺). In one embodiment, the salt of Formula I-A-X has the Formula I-A-01:

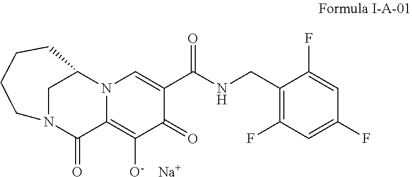 [SMILES: O=C(NCc1c(F)cc(F)cc1F)c1cn2c(c([O-])c1=O)C(=O)N1CCCC[C@H]2C1 ; [Na+]]

In one embodiment, n is 2 and X is calcium (Ca²⁺). In one embodiment, the salt of Formula I-A-X has the Formula I-A-02:

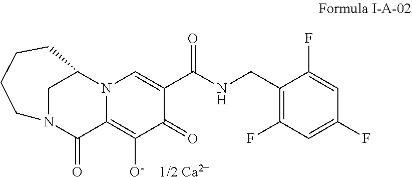

A salt of Formula I-A-01 can be prepared by reacting a compound of Formula I-A:

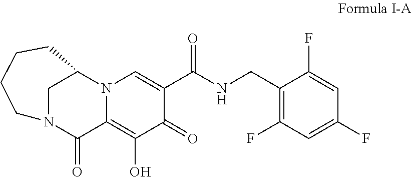 [SMILES: O=C(NCc1c(F)cc(F)cc1F)c1cn2c(c(O)c1=O)C(=O)N1CCCC[C@H]2C1]

or a salt thereof in the presence of a sixth base.

In some embodiments, the sixth base is a hydroxide, a carbonate, a bicarbonate, phosphate, a metal hydride, an alkoxide, or an amide. In some embodiments, the sixth base is a hydroxide. In some embodiments, the sixth base is sodium hydroxide. In some embodiments, the sixth base is a carbonate. In some embodiments, the sixth base is sodium carbonate. In some embodiments, the sixth base is a bicarbonate. In some embodiments, the sixth base is sodium bicarbonate. In some embodiments, the sixth base is a phosphate. In some embodiments, the sixth base is sodium phosphate. In some embodiments, the sixth base is a metal hydride. In some embodiments, the sixth base is sodium hydride. In some embodiments, the sixth base is an alkoxide. In some embodiments, the sixth base is sodium methoxide, sodium ethoxide, sodium tert-butoxide, or a combination thereof. In some embodiments, the sixth base is an amide. In some embodiments, the sixth base is sodium bis(trimethylsilyl)amide). In some embodiments, the sixth base is sodium hydroxide, sodium carbonate, sodium bicarbonate, sodium phosphate, sodium hydride, sodium methoxide, sodium ethoxide, sodium tert-butoxide, or sodium bis(trimethylsilyl)amide).

In some embodiments, the compound of Formula I-A is reacted with the sixth base further in the presence of a tenth solvent.

In some embodiments, the tenth solvent is an alcohol, an ether, a halogenated solvent, a hydrocarbon, a nitrile, a polar aprotic solvent, water, or a combination thereof. In some embodiments, the tenth solvent is an alcohol. In some embodiments, the tenth solvent is methanol, ethanol, 2-propanol, or a combination thereof. In some embodiments, the tenth solvent is an ether. In some embodiments, the tenth solvent is tetrahydrofuran, 2-methyltetrahydrofuran, tert-butyl methyl ether, or a combination thereof. In some embodiments, the tenth solvent is a halogenated solvent. In some embodiments, the tenth solvent is dichloromethane, 1,2-dichloroethane, chlorobenzene, or a combination thereof. In some embodiments, the tenth solvent is a hydrocarbon. In some embodiments, the tenth solvent is toluene, heptane (e.g., n-heptane), or a combination thereof. In some embodiments, the tenth solvent is a nitrile. In some embodiments, the tenth solvent is acetonitrile. In some embodiments, the tenth solvent is a polar aprotic solvent. In some embodiments, the tenth solvent is N,N-dimethylformamide, N,N-dimethylacetamide, N-methyl-2-pyrrolidone, or a combination thereof. In some embodiments, the tenth solvent is water. In some embodiments, the tenth solvent is ethanol, water, or a combination thereof. In some embodiments, the tenth solvent is a combination of ethanol and water.

In some embodiments, the compound of Formula I-A is reacted with the sixth base at a temperature of about 20 to about 110° C. In some embodiments, the compound of Formula I-A is reacted with the sixth base at a temperature of about 65 to about 85° C. In some embodiments, the compound of Formula I-A is reacted with the sixth base at a temperature of about 75° C.

In some embodiments, the sixth base is sodium hydroxide, the tenth solvent is ethanol, water, or a combination thereof and the temperature is about 65 to about 85° C. In some embodiments, the sixth base is sodium hydroxide, the tenth solvent is ethanol, water, or a combination thereof and the temperature is about 75° C.

A salt of Formula I-A-02 can be prepared by reacting a compound of Formula I-A:

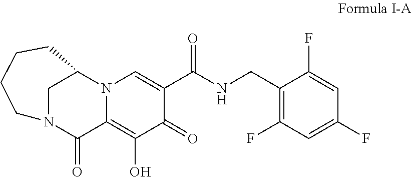 [SMILES: O=C(NCc1c(F)cc(F)cc1F)c1cn2c(c(O)c1=O)C(=O)N1CCCC[C@H]2C1]

or a salt thereof in the presence of a seventh base and a calcium source.

In some embodiments, the seventh base is a hydroxide, a carbonate, a bicarbonate, an alkoxide, an amine, a disilazane, or an organolithium reagent. In some embodiments, the seventh base is a hydroxide. In some embodiments, the seventh base is potassium hydroxide, lithium hydroxide, or sodium hydroxide. In some embodiments, the seventh base is potassium hydroxide. In some embodiments, the seventh base is a carbonate. In some embodiments, the seventh base is lithium carbonate, sodium carbonate, or potassium carbonate. In some embodiments, the seventh base is a bicarbonate. In some embodiments, the seventh base is lithium bicarbonate, sodium bicarbonate, or potassium bicarbonate. In some embodiments, the seventh base is an alkoxide. In some embodiments, the seventh base is sodium ethoxide, lithium tert-butoxide, sodium tert-butoxide, potassium tert-butoxide, or potassium tert-pentoxide. In some embodiments, the seventh base is an amine. In some embodiments, the seventh base is triethylamine, pyridine, or DBU. In some embodiments, the seventh base is a disilazane. In some embodiments, the seventh base is lithium bis(trimethylsilyl)amide, sodium bis(trimethylsilyl)amide, or potassium bis(trimethylsilyl)amide. In some embodiments, the seventh base is an organolithium reagent. In some embodiments, the seventh base is butyl lithium, or hexyl lithium. In some embodiments, the seventh base is triethylamine.

In some embodiments, the calcium source is a calcium salt. In some embodiments, the calcium salt is calcium acetate, calcium formate, calcium trifluoromethanesulfonate, calcium chloride, calcium bromide, calcium iodide, calcium carbonate, calcium hydroxide, calcium oxide, calcium hydride, calcium phosphate tribasic, calcium phosphate dibasic, calcium methoxide, calcium gluconate, calcium nitrate, or a combination thereof. In some embodiments, the calcium salt is calcium chloride.

In some embodiments, the compound of Formula I-A is reacted with the seventh base and the calcium source further in the presence of an eleventh solvent.

In some embodiments, the eleventh solvent is an alcohol, an ether, an ester, a halogenated solvent, a hydrocarbon, a nitrile, a polar aprotic solvent, a nitrile, water, or a combination thereof. In some embodiments, the eleventh solvent is an alcohol. In some embodiments, the eleventh solvent is methanol, ethanol, 2-propanol, or a combination thereof. In some embodiments, the eleventh solvent is an ether. In some embodiments, the eleventh solvent is tetrahydrofuran, 2-methyltetrahydrofuran, tert-butyl methyl ether, or a combination thereof. In some embodiments, the eleventh solvent is an ester. In some embodiments, the eleventh solvent is ethyl acetate or isopropyl acetate. In some embodiments, the eleventh solvent is a halogenated solvent. In some embodiments, the eleventh solvent is dichloromethane, 1,2-dichloroethane, chlorobenzene, or a combination thereof. In some embodiments, the eleventh solvent is a hydrocarbon. In some embodiments, the eleventh solvent is toluene or cyclohexane. In some embodiments, the eleventh solvent is a polar aprotic solvent. In some embodiments, the eleventh solvent is N,N-dimethylformamide, N,N-dimethylacetamide, N-methyl-2-pyrrolidone, or a combination thereof. In some embodiments, the eleventh solvent is a nitrile. In some embodiments, the eleventh solvent is acetonitrile. In some embodiments, the eleventh solvent is water. In some embodiments, the eleventh solvent is ethanol, water, or a combination thereof. In some embodiments, the eleventh solvent is a combination of ethanol and water. In some embodiments, the eleventh solvent is methanol, water, or a combination thereof. In some embodiments, the eleventh solvent is a combination of methanol and water. In some embodiments, the eleventh solvent is methanol, tetrahyrofuran, water, or a combination thereof. In some embodiments, the eleventh solvent is a combination of methanol, tetrahyrofuran, and water.

In some embodiments, the compound of Formula I-A is reacted with the seventh base and the calcium source at a temperature of about 5 to about 90° C. In some embodiments, the compound of Formula I-A is reacted with the seventh base and the calcium source at a temperature of about 10 to about 30° C. In some embodiments, the compound of Formula I-A is reacted with the seventh base and the calcium source at a temperature of about 25° C.

In some embodiments, the seventh base is potassium hydroxide, the calcium source is calcium chloride, the eleventh solvent is ethanol, water, or a combination thereof and the temperature is about 10 to about 30° C. In some embodiments, the seventh base is potassium hydroxide, the calcium source is calcium chloride, the eleventh solvent is ethanol, water, or a combination thereof and the temperature is about 25° C.

In some embodiments, the seventh base is triethylamine, the calcium source is calcium chloride, the eleventh solvent is methanol, water, or a combination thereof and the temperature is about 10 to about 30° C. In some embodiments, the seventh base is triethylamine, the calcium source is calcium chloride, the eleventh solvent is methanol, water, or a combination thereof and the temperature is about 25° C.

In some embodiments, the seventh base is triethylamine, the calcium source is calcium hydroxide, the eleventh solvent is methanol, tetrahydrofuran, water, or a combination thereof, and the temperature is about 10 to about 30° C. In some embodiments, the seventh base is triethylamine, the calcium source is calcium hydroxide, the eleventh solvent is methanol, tetrahydrofuran, water, or a combination thereof, and the temperature is about 25° C.

In some embodiments, the reacting of the compound of Formula I-A or a salt thereof in the presence of a seventh base and a calcium source provides a mixture comprising a compound of Formula I-A-02 and an additional calcium salt. In some embodiments, the additional calcium salt is a calcium salt of chloride, such as CaCl⁺ or CaCl₂. In some embodiments, the mixture comprises about 0.1 to about 3.0 wt %, about 0.3 to about 2.5 wt %, about 0.4 to about 2.0 wt %, about 0.5 to about 1.0 wt %, about 0.6 to about 0.9 wt %, about 0.6 wt %, about 0.7 wt %, about 0.8 wt %, or about 0.9 wt % of chloride.

In some embodiments, the reacting of the compound of Formula I-A or a salt thereof in the presence of a seventh base and a calcium source provides a mixture comprising a compound of Formula I-A-02 and the compound of Formula I in free form. In some embodiments, the mixture further comprises chloride.

In some embodiments a process for preparing a compound of Formula I-A:

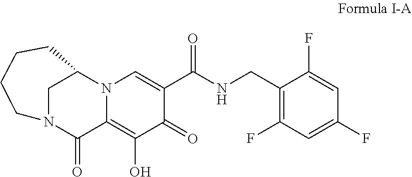 [SMILES: O=C(NCc1c(F)cc(F)cc1F)c1cn2c(c(O)c1=O)C(=O)N1CCCC[C@H]2C1]

or a salt thereof is provided, comprising treating a compound of Formula A:

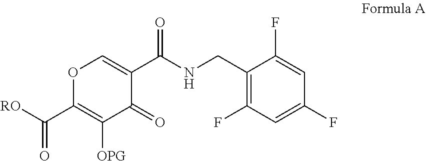

with a compound of Formula B-A:

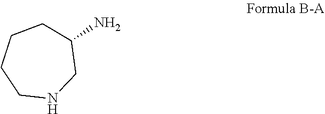

or a salt thereof, wherein PG is a hydroxyl protecting group and R is C₁₋₆ alkyl, C₆₋₁₀ aryl-C₁₋₄ alkylene, or C₆₋₁₀ aryl.

In some embodiments, the treatment of the compound of Formula A with a compound of Formula B-A or a salt thereof provides a compound of Formula C-A:

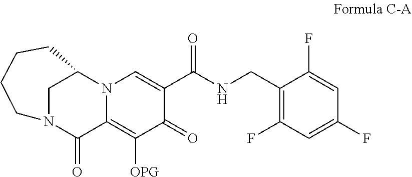

wherein deprotection of the compound of Formula C-A provides a compound of Formula I-A or a salt thereof.

In some embodiments, provided herein are intermediates for the formation of a compound of Formula I (e.g., a compound of Formula I-A) or salts thereof.

Accordingly, in some embodiments, a compound of Formula A:

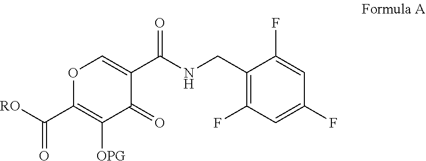

is provided, wherein PG is a hydroxyl protecting group and R is C₁₋₆ alkyl, C₆₋₁₀ aryl-C₁₋₄ alkylene, or C₆₋₁₀ aryl. In one embodiment, the compound of Formula A is a compound of formula (1):

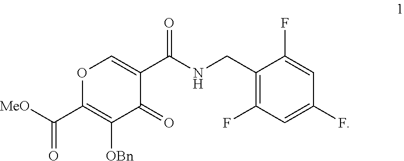 [SMILES: COC(=O)c1occ(C(=O)NCc2c(F)cc(F)cc2F)c(=O)c1OCc1ccccc1]

In one embodiment, the compound of Formula A is a compound of formula (4a):

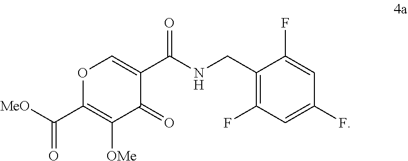 [SMILES: COC(=O)c1occ(C(=O)NCc2c(F)cc(F)cc2F)c(=O)c1OC]

In one embodiment, the compound of Formula A is a compound of formula (4b):

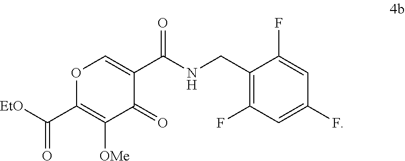 [SMILES: CCOC(=O)c1occ(C(=O)NCc2c(F)cc(F)cc2F)c(=O)c1OC]

In some embodiments, a compound of Formula B:

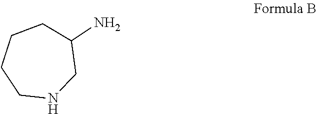

or a salt thereof is provided. In one embodiment, the compound of Formula B is a compound of Formula B-A:

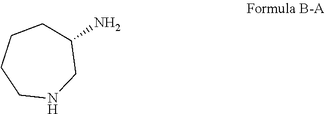

or a salt thereof.

In some embodiments, a compound of Formula C:

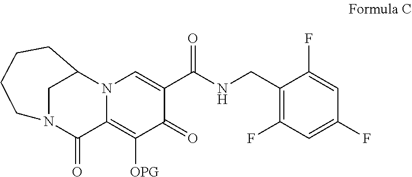

is provided, wherein PG is a hydroxyl protecting group. In one embodiment, the compound of Formula C is a compound of Formula C-A:

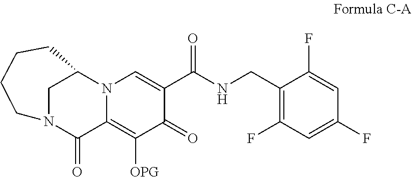

wherein PG is a hydroxyl protecting group. In one embodiment, the compound of Formula C-A is a compound of formula (2):

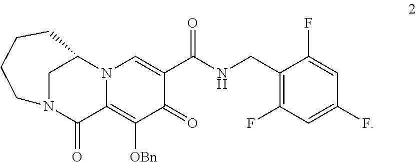 [SMILES: O=C(NCc1c(F)cc(F)cc1F)c1cn2c(c(OCc3ccccc3)c1=O)C(=O)N1CCCC[C@H]2C1]

In one embodiment, the compound of Formula C-A is a compound of formula (5):

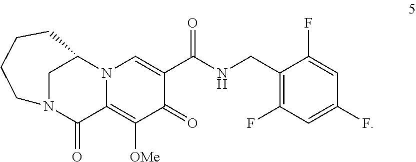 [SMILES: COc1c2n(cc(C(=O)NCc3c(F)cc(F)cc3F)c1=O)[C@H]1CCCCN(C1)C2=O]

In some embodiments, a compound of Formula D:

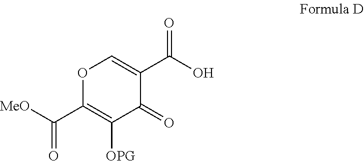

is provided, wherein PG is a hydroxyl protecting group. In one embodiment, the compound of Formula D is a compound of formula (1a):

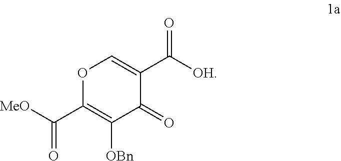 [SMILES: COC(=O)c1occ(C(=O)O)c(=O)c1OCc1ccccc1]

In some embodiments, a compound of Formula E:

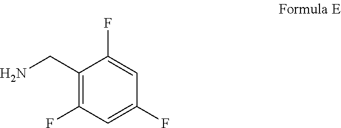 [SMILES: NCc1c(F)cc(F)cc1F]

or a salt thereof is provided.

In some embodiments, a compound of Formula F:

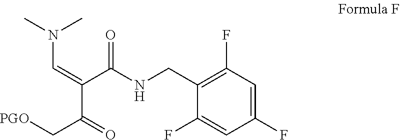

is provided, wherein PG is a hydroxyl protecting group. In one embodiment, the compound of Formula F is a compound of formula (4):

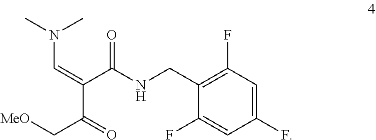 [SMILES: COCC(=O)/C(=C/N(C)C)C(=O)NCc1c(F)cc(F)cc1F]

In one embodiment, the compound of Formula F is a compound of formula (8):

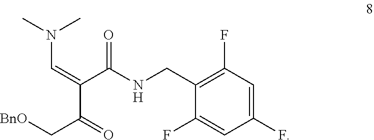 [SMILES: CN(C)/C=C(/C(=O)COCc1ccccc1)C(=O)NCc1c(F)cc(F)cc1F]

In some embodiments, a compound of Formula G:

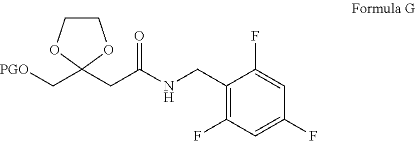

is provided, wherein PG is a hydroxyl protecting group. In one embodiment, the compound of Formula G is a compound of formula (7):

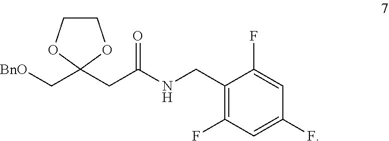 [SMILES: O=C(CC1(COCc2ccccc2)OCCO1)NCc1c(F)cc(F)cc1F]

In some embodiments, a compound of Formula H:

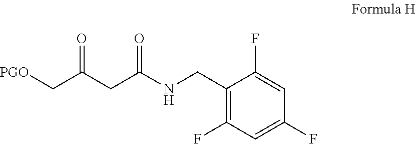

is provided, wherein PG is a hydroxyl protecting group. In one embodiment, the compound of Formula H is a compound of formula (8a):

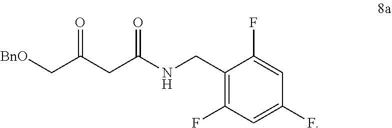 [SMILES: O=C(COCc1ccccc1)CC(=O)NCc1c(F)cc(F)cc1F]

In some embodiments, a compound of Formula J:

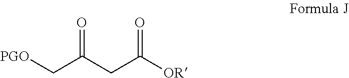

is provided, wherein PG is a hydroxyl protecting group and R′ is C₁₋₆ alkyl, C₆₋₁₀ aryl-C₁₋₄ alkylene, or C₆₋₁₀ aryl. In one embodiment, the compound of Formula J is a compound of formula (7a):

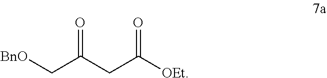 [SMILES: CCOC(=O)CC(=O)COCc1ccccc1]

In some embodiments, a compound of Formula L:

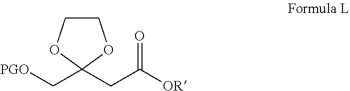

is provided, wherein PG is a hydroxyl protecting group. In one embodiment, the compound of Formula L is a compound of formula (7b):

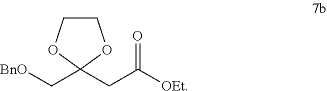 [SMILES: CCOC(=O)CC1(COCc2ccccc2)OCCO1]

In some embodiments, a compound of Formula M:

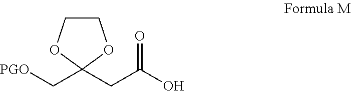

is provided, wherein PG is a hydroxyl protecting group. In one embodiment, the compound of Formula M is a compound of formula (7c):

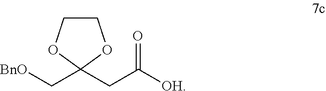 [SMILES: O=C(O)CC1(COCc2ccccc2)OCCO1]

### EXAMPLES

Representative syntheses of compounds of the present disclosure are described in schemes below, and the particular examples that follow. The following examples are merely illustrative, and not intended to limit this disclosure in any way. It is to be understood that individual steps described herein may be combined. It is also to be understood that separate batches of a compound may be combined and carried forth in the next synthetic step.

### Example 1: Preparation of methyl 3-(benzyloxy)-4-oxo-5-((2,4,6-trifluorobenzyl)carbamoyl)-4H-pyran-2-carboxylate (1) from 5-(benzyloxy)-6-(methoxycarbonyl)-4-oxo-4H-pyran-3-carboxylic acid (1a)

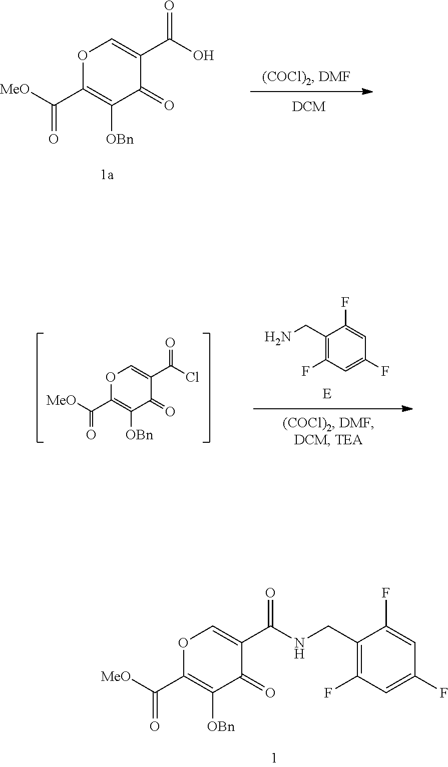

5-(Benzyloxy)-6-(methoxycarbonyl)-4-oxo-4H-pyran-3-carboxylic acid (1a, 1.00 equiv, scaling factor) and dichloromethane (15.0 volumes) were charged to a reactor and cooled to about −5° C. Oxalyl chloride (1.5 equiv) was charged, followed by N,N-dimethylformamide (0.1 volumes). The mixture was agitated at about −5° C. until the reaction was deemed complete. The mixture was warmed to about 25° C. and concentrated under reduced pressure. Dichloromethane (1.1 volumes) was charged, and the mixture was cooled to about −10° C. To a separate vessel was charged (2,4,6-trifluorophenyl)methanamine (E, 1.2 equiv), DCM (4.5 volumes), and triethylamine (1.7 equiv). The resulting solution was charged to the reaction mixture, which was agitated until the reaction was deemed complete. The reaction mixture was concentrated partially under reduced pressure. The reaction mixture was washed with 1 M HCl (15 volumes), washed twice more with 1 M HCl (12.5 volumes), and finally washed with 5% aq NaCl (15 volumes). The mixture was concentrated under reduced pressure and MTBE (10 volumes) was charged. The mixture was concentrated again under reduced pressure and additional MTBE (5 volumes) was charged. The mixture was heated to about 40° C. for about 1 h, cooled to about 15° C. for about 1 h, heated again to about 40° C. for about 1 h, and final cooled to −5° C. and aged for about 2 hours. The slurry was filtered, and the cake rinsed twice with MTBE (3.0 volumes), and dried to give methyl 3-(benzyloxy)-4-oxo-5-((2,4,6-trifluorobenzyl)carbamoyl)-4H-pyran-2-carboxylate (1). ¹H NMR (400 MHz, CDCl₃): δ 9.44 (s, 1H), 8.78 (s, 1H), 7.57-7.42 (m, 2H), 7.41-7.29 (m, 3H), 6.82-6.57 (m, 2H), 5.27 (s, 2H), 4.66 (d, J=5.6 Hz, 2H), 3.88 (s, 3H).

### Example 2: Preparation of (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A) from methyl 3-(benzyloxy)-4-oxo-5-((2,4,6-trifluorobenzyl)carbamoyl)-4H-pyran-2-carboxylate (1)

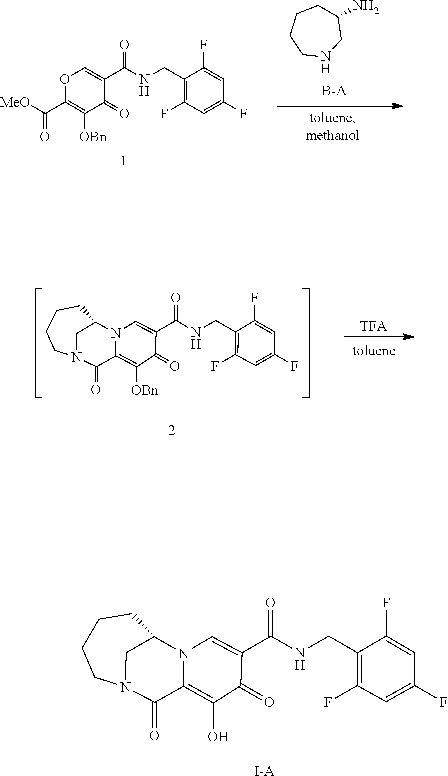

Methyl 3-(benzyloxy)-4-oxo-5-((2,4,6-trifluorobenzyl)carbamoyl)-4H-pyran-2-carboxylate (1, 1.0 equiv, scaling factor), toluene (5.4 volumes), (S)-azepan-3-amine (B-A, 1.1 equiv.), and methanol (0.6 volumes) were charged to a reactor. The mixture was agitated at about 55° C. until the reaction was deemed complete. The mixture was concentrated under reduced pressure. Toluene (4.0 volumes) was charged, and the mixture was concentrated again under reduced pressure. Toluene (4.0 volumes) and trifluoroacetic acid (5.0 equiv) were charged, and the mixture was agitated at about 55° C. until the reaction was deemed complete. The mixture was concentrated under reduced pressure. Toluene (4.0 volumes) was charged, and the mixture was again concentrated under reduced pressure. 2-Propanol (2.0 volumes) was charged, and the mixture was agitated at about 55° C. for about 1 hour. Additional 2-propanol (10.0 volumes) was charged over about 1 hour, the mixture was cooled to about 25° C. and agitated for about 16 hours. The slurry was filtered and resulting cake rinsed with 2-propanol (3.0 volumes), rinsed twice with ethanol (3.0 volumes), dried to afford (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A). ¹H NMR (400 MHz, CDCl₃): δ 10.8 (br s, 1H), 10.35 (s, 1H), 8.38 (s, 1H), 6.72-6.65 (dd, J=8.0, 12.0 Hz, 2H), 4.75-4.62 (m, 2H), 4.45-4.36 (m, 2H), 3.97 (dt, J=4.0, 14.8 Hz, 1H), 3.52 (dd, J=4.0, 16.0 Hz, 1H), 3.20-3.12 (m, 1H), 2.19-1.97 (m, 3H), 1.87-1.75 (m, 2H), 1.44-1.31 (m, 1H).

### Example 3: Preparation of Sodium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-01)

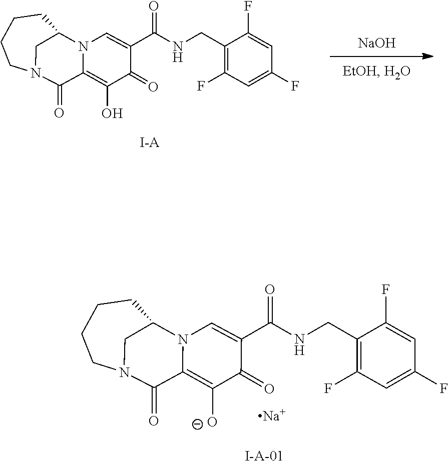

(7S)-12-Hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A, 1.00 equiv, scaling factor) and ethanol (10.0 volumes) were charged to a reactor. The mixture was heated to about 75° C. and 25 wt % sodium hydroxide in water (1.1 equiv) was charged to the reactor, followed by rinsing forward with water (0.2 volumes). The reaction mixture was agitated for about 0.5 h, cooled to about 20° C. over about 4 h, and agitated for about 18 hours. The slurry was filtered, rinsed twice with 95% ethanol in water (2.0 volumes), and dried to afford sodium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-01). ¹H NMR (400 MHz, DMSO-d6): δ 10.77 (t, J=5.7 Hz, 1H), 7.89 (s, 1H), 7.16 (dd, J=9.2, 7.9 Hz, 2H), 4.51 (d, J=5.8 Hz, 3H), 4.17 (dt, J=14.1, 7.4 Hz, 1H), 3.68 (d, J=14.3 Hz, 1H), 3.54 (d, J=13.4 Hz, 1H), 2.89 (dd, J=11.3, 5.7 Hz, 1H), 2.13-1.92 (m, 1H), 1.90-1.65 (m, 3H), 1.65-1.46 (m, 1H), 1.21 (q, J=11.7 Hz, 1H).

### Example 4a: Preparation of methyl 3-methoxy-4-oxo-5-((2,4,6-trifluorobenzyl)carbamoyl)-4H-pyran-2-carboxylate (4a)

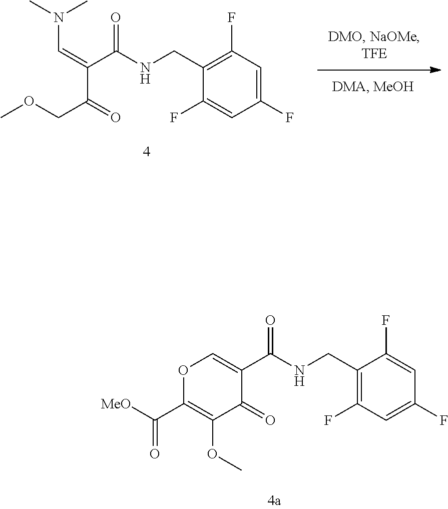 [SMILES: COCC(=O)/C(=C/N(C)C)C(=O)NCc1c(F)cc(F)cc1F ; COC(=O)c1occ(C(=O)NCc2c(F)cc(F)cc2F)c(=O)c1OC]

(Z)-2-((Dimethylamino)methylene)-4-methoxy-3-oxo-N-(2,4,6-trifluorobenzyl) butanamide (4, 1.00 equiv, scaling factor), dimethyl oxalate (5.0 equiv), and N,N-dimethylacetamide (10.0 volumes) were charged to a reactor. The mixture was cooled to about 10° C. and sodium methoxide in methanol (2.0 equiv) was charged slowly and agitated for about 1 hour. 2,2,2-trifluoroethanol (5.0 equiv) was charged slowly to the reactor and agitated for about 23 hours until the reaction was deemed complete. Methanol (5.0 volumes) was charged slowly and agitated at about 20° C. for about 15 min. 1 N aqueous hydrochloric acid (2.5 equiv) was charged over about 1 h to the reactor. The slurry was agitated for about 3 h at 20° C. The slurry was filtered, rinsed with 50% methanol in water (10.0 volumes), and dried to afford methyl 3-methoxy-4-oxo-5-((2,4,6-trifluorobenzyl)carbamoyl)-4H-pyran-2-carboxylate (4a). 1H NMR (400 MHz, CDCl₃): δ 9.39 (br s, 1H), 8.77 (s, 1H), 6.68 (dd, J=8.7, 7.5 Hz, 2H), 4.64 (d, J=5.7 Hz, 2H), 4.01 (s, 3H), 3.97 (s, 3H).

### Example 4b: Preparation of ethyl 3-methoxy-4-oxo-5-((2,4,6-trifluorobenzyl)carbamoyl)-4H-pyran-2-carboxylate (4b)

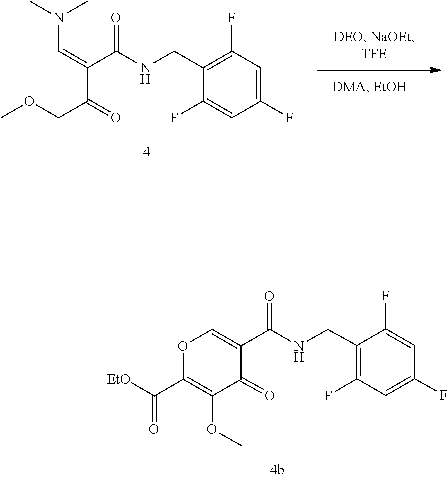 [SMILES: COCC(=O)/C(=C/N(C)C)C(=O)NCc1c(F)cc(F)cc1F ; CCOC(=O)c1occ(C(=O)NCc2c(F)cc(F)cc2F)c(=O)c1OC]

(Z)-2-((Dimethylamino)methylene)-4-methoxy-3-oxo-N-(2,4,6-trifluorobenzyl) butanamide (4, 1.00 equiv, scaling factor), diethyl oxalate (5.0 equiv), and N,N-dimethylacetamide (7.0 volumes) were charged to a reactor. The mixture was cooled to about 0° C. and sodium ethoxide in ethanol (1.5 equiv) was charged slowly and agitated for about 1 h. 2,2,2-Trifluoroethanol (5.0 equiv) was charged to the reactor and the mixture warmed to about 10° C. and agitated for about 24 hours until the reaction was deemed complete. The mixture was cooled to about 0° C. then 2-ethylhexanoic acid (0.65 equiv) was charged followed by ethyl 3-methoxy-4-oxo-5-((2,4,6-trifluorobenzyl)carbamoyl)-4H-pyran-2-carboxylate (4b) seed, if available, and aged for about 16 h. Additional 2-ethylhexanoic acid (0.7 equiv) was charged over about 3 hours, followed by a final portion of 2-ethylhexanoic acid (0.65 equiv) over about 30 minutes. The resulting mixture was agitated for about 20 hours at about 0° C. then filtered, cake rinsed twice with ethanol (3.0 volumes), and dried to afford ethyl 3-methoxy-4-oxo-5-((2,4,6-trifluorobenzyl)carbamoyl)-4H-pyran-2-carboxylate (4b). ¹H NMR (400 MHz, CDCl₃): δ 9.40 (s, 1H), 8.76 (s, 1H), 6.74-6.58 (m, 2H), 4.62 (d, J=5.8 Hz, 2H), 4.42 (q, J=7.1 Hz, 2H), 3.98 (s, 3H), 1.40 (t, J=7.1 Hz, 3H).

### Example 5a: Preparation of (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (5) from methyl 3-methoxy-4-oxo-5-((2,4,6-trifluorobenzyl)carbamoyl)-4H-pyran-2-carboxylate (4a)

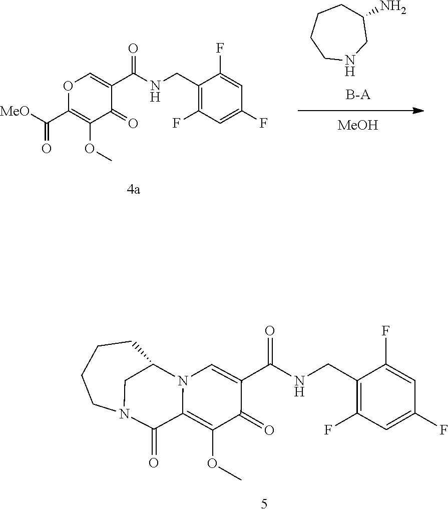 [SMILES: "MeOH" ; COC(=O)c1occ(C(=O)NCc2c(F)cc(F)cc2F)c(=O)c1OC ; N[C@H]1CCCCNC1 ; COc1c2n(cc(C(=O)NCc3c(F)cc(F)cc3F)c1=O)[C@H]1CCCCN(C1)C2=O]

Methyl 3-methoxy-4-oxo-5-((2,4,6-trifluorobenzyl)carbamoyl)-4H-pyran-2-carboxylate (4a, 1.00 equiv, scaling factor), (S)-azepan-3-amine (B-A, 1.10 equiv), and methanol (10.0 volumes) were charged to a reactor. The mixture was agitated at about 50° C. for about 3 hours until the reaction was deemed complete. To the reactor was added water (10.0 volumes) and (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide seed (5, 0.001 equiv), if available, and agitated at about 50° C. for about 1 hour. Additional water (10.0 volumes) was added at about 50° C., and the mixture cooled to about 20° C. and agitated for about 16 hours. The slurry was filtered and the cake rinsed with 20% methanol in water. The obtained solids were dried at about 50° C. to afford (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (5). ¹H NMR (400 MHz, CDCl₃): δ 10.53-10.33 (m, 1H), 8.45 (s, 1H), 6.79-6.54 (m, 2H), 4.75-4.58 (m, 2H), 4.57-4.44 (m, 1H), 4.38 (dd, J=5.5, 2.7 Hz, 1H), 4.07 (s, 3H), 3.91 (dt, J=14.8, 2.2 Hz, 1H), 3.47 (dd, J=14.7, 1.9 Hz, 1H), 3.01 (ddd, J=13.6, 7.2, 3.3 Hz, 1H), 2.22-1.95 (m, 3H), 1.83-1.67 (m, 2H), 1.50-1.33 (m, 1H).

### Example 5b: Preparation of (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (5) from ethyl 3-methoxy-4-oxo-5-((2,4,6-trifluorobenzyl)carbamoyl)-4H-pyran-2-carboxylate (4b)

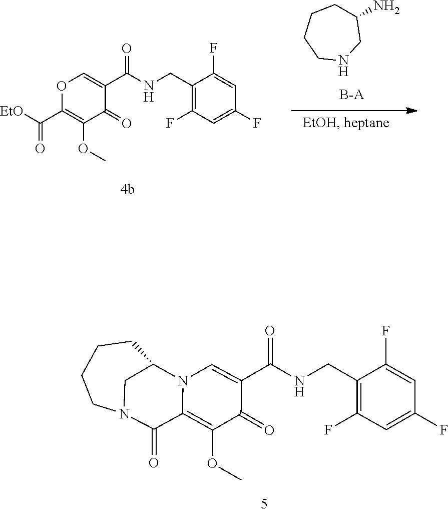 [SMILES: CCOC(=O)c1occ(C(=O)NCc2c(F)cc(F)cc2F)c(=O)c1OC ; N[C@H]1CCCCNC1 ; "EtOH, heptane" ; COc1c2n(cc(C(=O)NCc3c(F)cc(F)cc3F)c1=O)[C@H]1CCCCN(C1)C2=O]

Ethyl 3-methoxy-4-oxo-5-((2,4,6-trifluorobenzyl)carbamoyl)-4H-pyran-2-carboxylate (4b, 1.00 equiv, scaling factor), ethanol (5.0 volumes), heptane (5.0 volumes), and (S)-azepan-3-amine (B-A, 1.10 equiv) were charged to a reactor. The mixture was agitated at about 70° C. for about 17 h. The mixture was cooled to about 0° C. over about 3 hours. Heptane (5.0 volumes) was charged over about 1 hour and the resulting mixture agitated for about 18 hours. The mixture was filtered, cake rinsed twice with 30% ethanol in heptane (3.0 volumes), and dried to afford (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (5). ¹H NMR (400 MHz, CDCl₃): δ 10.53-10.33 (m, 1H), 8.45 (s, 1H), 6.79-6.54 (m, 2H), 4.75-4.58 (m, 2H), 4.57-4.44 (m, 1H), 4.38 (dd, J=5.5, 2.7 Hz, 1H), 4.07 (s, 3H), 3.91 (dt, J=14.8, 2.2 Hz, 1H), 3.47 (dd, J=14.7, 1.9 Hz, 1H), 3.01 (ddd, J=13.6, 7.2, 3.3 Hz, 1H), 2.22-1.95 (m, 3H), 1.83-1.67 (m, 2H), 1.50-1.33 (m, 1H).

### Example 6: Preparation of (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A) from (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (5)

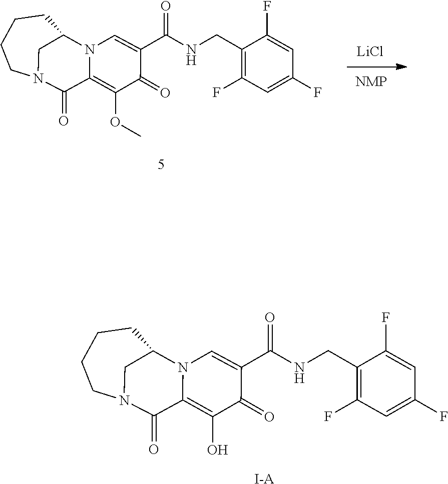

Charged (7S)-12-methoxy-1,11-dioxo-N-(2,4,6-trifluorobenzyl)-1,4,5,6,7,11-hexahydro-3H-2,7-methanopyrido[1,2-a][1,4]diazonine-10-carboxamide (5, 1.00 equiv, scaling factor), lithium chloride (3.0 equiv), and N-methyl-2-pyrrolidone (3.0 volumes) to a reactor. The mixture was heated to about 100° C. and agitated until the reaction was deemed complete. The reaction mixture was charged to another reactor containing 1 M hydrochloric acid (3.0 volumes) at about 2° C. The slurry was agitated at about 25° C. for about 16 hours. The slurry was filtered and rinsed twice with water (3.0 volumes), rinsed twice with 2-propanol (3.0 volumes), and dried to afford (7S)-12-hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A). ¹H NMR (400 MHz, CDCl₃): δ 10.8 (br s, 1H), 10.35 (s, 1H), 8.38 (s, 1H), 6.72-6.65 (dd, J=8.0, 12.0 Hz, 2H), 4.75-4.62 (m, 2H), 4.45-4.36 (m, 2H), 3.97 (dt, J=4.0, 14.8 Hz, 1H), 3.52 (dd, J=4.0, 16.0 Hz, 1H), 3.20-3.12 (m, 1H), 2.19-1.97 (m, 3H), 1.87-1.75 (m, 2H), 1.44-1.31 (m, 1H).

### Example 7: Preparation of 2-(2-((benzyloxy)methyl)-1,3-dioxolan-2-yl)-N-(2,4,6-trifluorobenzyl)acetamide (7)

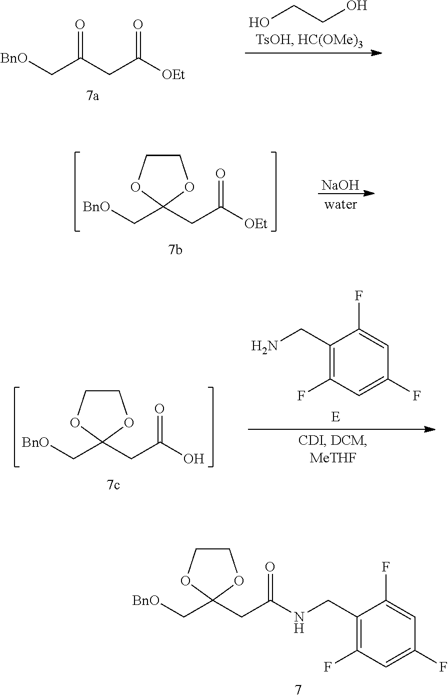

Ethyl 4-(benzyloxy)-3-oxobutanoate (7a, scaling factor, 1 equiv), ethylene glycol (1.05 equiv), trimethyl orthoformate (1.25 equiv), and p-toluenesulfonic acid monohydrate (0.1 equiv) were charged to a reactor. The resulting mixture was agitated at about 40° C. for about 3 hours until the reaction was deemed complete, concentrated to dryness under vacuum. Sodium hydroxide (4N, 1.25 equiv) was charged and the mixture was agitated for about 15 hours until the reaction was deemed complete. The pH was adjusted to about 1.5 with 1M aqueous HCl, extracted into 2methyltetrahydrofuran (3.0 volumes), and the organic layer was dried over MgSO₄ concentrated to dryness under vacuum. 2-Methyltetrahydrofuran (3.0 volumes) and carbonyl diimidazole (1.0 equiv) were charged and the mixture was agitated for about 30 minutes. A solution of (2,4,6-trifluorophenyl)methanamine (E, 1.0 equiv) in 2-methyltetrahydrofuran (1.5 volumes) was charged and the mixture was agitated for about 15 hours until the reaction was deemed complete. The mixture was washed with water (3.0 volumes) and twice with aqueous HCl (1M, 3.0 volumes each). The organic layer was dried over MgSO₄, concentrated to dryness under vacuum. The residue was dissolved in 2-methyltetrahydrofuran (3.0 volumes) and crystallized by adding hexanes (9.0 volumes). The slurry was filtered, the filter cake was washed with 25% solution of 2-methyltetrahydrofuran in hexanes (4.0 volumes), and dried to afford 2-(2-((benzyloxy)methyl)-1,3-dioxolan-2-yl)-N-(2,4,6-trifluorobenzyl)acetamide (7). ¹H NMR (300 MHz, CDCl₃): δ 7.35-7.27 (m, 5H), 6.65 (t, J=8.3 Hz, 2H), 6.57 (br t, 1H), 4.54 (s, 2H), 4.44 (d, J=5.1 Hz, 2H), 4.01-3.90 (m, 4H), 3.42 (s, 2H), 2.69 (s, 2H).

### Example 8: Preparation of (Z)-4-(benzyloxy)-2-((dimethylamino)methylene)-3-oxo-N-(2,4,6-trifluorobenzyl) butanamide (8)

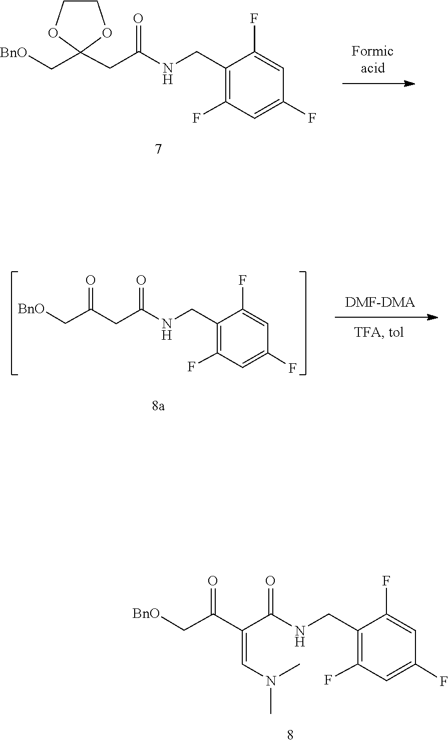

2-(2-((Benzyloxy)methyl)-1,3-dioxolan-2-yl)-N-(2,4,6-trifluorobenzyl)acetamide (7, scaling factor, 1 equiv) and formic acid (20 equiv) were charged to a reactor. The resulting mixture was agitated at about 65° C. for about 18 hours until the reaction was deemed complete. Toluene (5.0 volumes) was charged, and the mixture was washed with saturated aqueous sodium chloride (5.0 volumes), saturated aqueous sodium bicarbonate (5.0 volumes) and saturated aqueous sodium chloride (5.0 volumes). The organic layer was dried over MgSO₄, concentrated to dryness under vacuum. The residue was dissolved in toluene (5.0 volumes) and N,N-dimethylformamide dimethyl acetal (1.5 equiv) and trifluoroacetic acid (0.1 equiv) were charged. The mixture was stirred at about 90° C. for about 18 hours until the reaction was deemed complete, cooled to about 20° C. and crystallized by adding heptane (15 volumes). The slurry was filtered, the filter cake was washed with 25% solution of toluene in heptane (4.0 volumes), and dried to afford (Z)-4-(benzyloxy)-2-((dimethylamino)methylene)-3-oxo-N-(2,4,6-trifluorobenzyl) butanamide (8). ¹H NMR (300 MHz, CDCl₃): δ 8.43 (br t, 1H), 7.89 (s, 1H), 7.33-7.28 (m, 5H), 6.63 (t, J=8.3 Hz, 2H), 4.55-4.53 (m, 4H), 4.22 (s, 2H), 3.07 (s, 6H).

### Example 9: Preparation of methyl 3-(benzyloxy)-4-oxo-5-((2,4,6-trifluorobenzyl)carbamoyl)-4H-pyran-2-carboxylate (1) from (Z)-4-(benzyloxy)-2-((dimethylamino)methylene)-3-oxo-N-(2,4,6-trifluorobenzyl)butanamide (8)

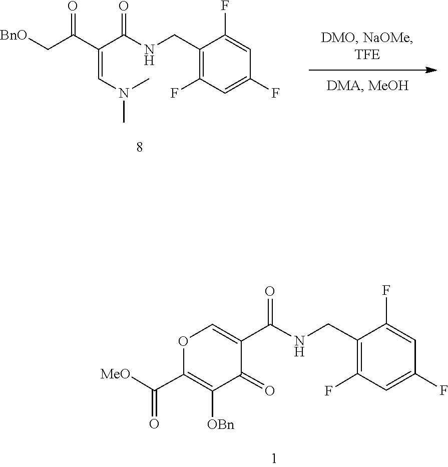 [SMILES: CN(C)/C=C(/C(=O)COCc1ccccc1)C(=O)NCc1c(F)cc(F)cc1F ; COC(=O)c1occ(C(=O)NCc2c(F)cc(F)cc2F)c(=O)c1OCc1ccccc1]

Charged (Z)-4-(benzyloxy)-2-((dimethylamino)methylene)-3-oxo-N-(2,4,6-trifluorobenzyl) butanamide (8, 1.00 equiv, scaling factor), dimethyl oxalate (5.0 equiv), and N,N-dimethylacetamide (10.0 volumes) to a reactor. Cooled to about 10° C. and charged sodium methoxide in methanol (2.0 equiv), and agitated for about 1 hour. Charged 2,2,2-trifluoroethanol (5.0 equiv) and agitated for about 23 hours until the reaction was deemed complete. Diluted with dichloromethane (10.0 volumes) and washed with 1 M HCl (15.0 volumes), twice with 1 M HCl (12.5 volumes), and 5% aqueous NaCl (15 volumes). Concentrated to dryness under vacuum, and added MTBE (5 volumes). Filtered the slurry and dried the filter cake to afford methyl 3-(benzyloxy)-4-oxo-5-((2,4,6-trifluorobenzyl)carbamoyl)-4H-pyran-2-carboxylate (1). ¹H NMR (400 MHz, CDCl₃): δ 9.44 (s, 1H), 8.78 (s, 1H), 7.57-7.42 (m, 2H), 7.41-7.29 (m, 3H), 6.82-6.57 (m, 2H), 5.27 (s, 2H), 4.66 (d, J=5.6 Hz, 2H), 3.88 (s, 3H).

### Example 10: Preparation of Calcium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-02)

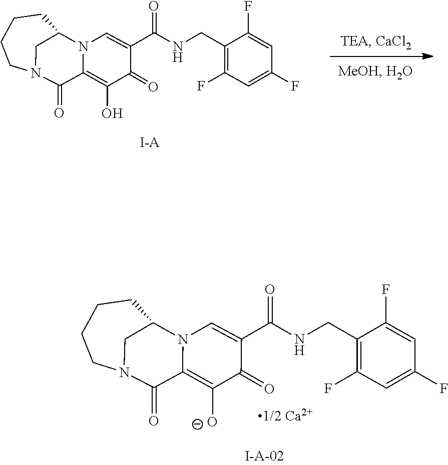

(7S)-12-Hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A, 1.00 equiv, scaling factor), methanol (6.0 volumes), and water (3.0 volumes) were charged to a reactor and stirred. Triethylamine (1.4 equiv.) was charged. The mixture was agitated for about 1 hour at room temperature, and polish filtered into a clean reactor. A 1M aq. calcium chloride solution (0.5 equiv.) was charged over about 3 hours. The mixture was agitated for about 6 hours. The slurry was filtered, and the solids rinsed with a 50 vol % solution of methanol in water (3.0 volumes) twice, and dried to afford calcium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-02). ¹H NMR (400 MHz, MeOD w/DCl): δ 8.71 (s, 1H), δ 6.90 (t, 2H), δ 4.87 (d, 1H), δ 4.26 (s, 2H), δ 4.30 (m, 1H), δ 4.05 (d, 1H), δ 3.81 (d, 1H), 3.25 (m, 1H), 2.25-1.75 (m, 5H), 1.27 (q, 1H).

### Example 11: Preparation of Calcium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-02)

Hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A, 1.00 equiv, scaling factor), ethanol (6.0 volumes), and water (2.5 volumes) were charged to a reactor and stirred. Potassium hydroxide (1.05 equiv., 50% w/v aq.) was charged. The mixture was agitated for about 24 hours at room temperature, and polish filtered into a clean reactor. Heptanes (2.0 volumes) were added, followed by 1M aq. calcium chloride solution (0.5 equiv.) charged over about 6 hours. The mixture was agitated for about 1 hour. The slurry was filtered, and the solids rinsed with water (5.0 volumes) twice, and dried to afford calcium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-02).

### Example 12: Preparation of Calcium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-02)

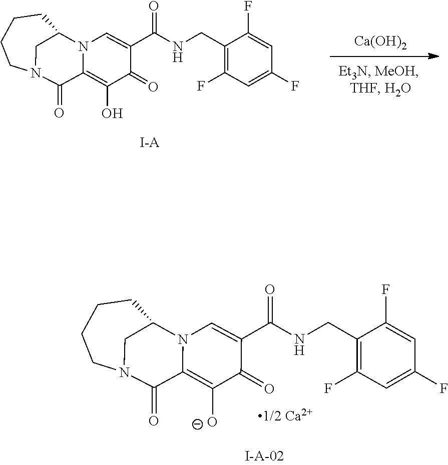

(7S)-12-Hydroxy-1,11-dioxo-N-(2,4,6-trifluorobenzyl)-1,4,5,6,7,11-hexahydro-3H-2,7-methanopyrido[1,2-a][1,4]diazonine-10-carboxamide (I-A, 1.00 equiv, scaling factor), calcium hydroxide (0.50 equiv.), methanol (4.0 volumes), tetrahydrofuran (4.0 volumes), triethylamine (2.0 equiv.) and water (1.0 volumes) were charged to a reactor and stirred. The mixture was agitated for about 18 hours at room temperature, then polish filtered into a clean reactor. Water (9.0 volumes) was added over about 2 hours, and the mixture was agitated for about 6 hours. The slurry was filtered, and the solids rinsed with a 20 vol % solution of methanol in water containing 1.0 vol % triethylamine (3.0 volumes) twice, and dried to afford calcium (7S)-1,11-dioxo-10-((2,4,6-trifluorobenzyl)carbamoyl)-1,4,5,6,7,11-hexahydro-3H-2,7-methanopyrido[1,2-a][1,4]diazonin-12-olate (I-A-02). ¹H NMR (400 MHz, MeOD w/DCl): δ 8.71 (s, 1H), δ 6.90 (t, 2H), δ 4.87 (d, 1H), δ 4.26 (s, 2H), δ 4.30 (m, 1H), δ 4.05 (d, 1H), δ 3.81 (d, 1H), 3.25 (m, 1H), 2.25-1.75 (m, 5H), 1.27 (q, 1H).

All references, including publications, patents, and patent documents were incorporated by reference herein, as though individually incorporated by reference. The present disclosure provides reference to various embodiments and techniques. However, it should be understood that many variations and modifications may be made while remaining within the spirit and scope of the present disclosure.

1. A method of preparing a compound of Formula I: 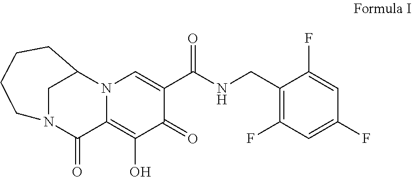 [SMILES: O=C(NCc1c(F)cc(F)cc1F)c1cn2c(c(O)c1=O)C(=O)N1CCCCC2C1]
or a salt thereof, the method comprising:
treating a compound of Formula A: 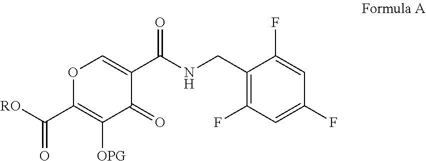
with a compound of Formula B 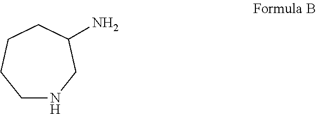
or a salt thereof;
wherein PG is a hydroxyl protecting group and R is C₁₋₆ alkyl, C₆₋₁₀ aryl-C₁₋₄ alkylene, or C₆₋₁₀ aryl.

2.-13. (canceled)

14. The method of claim 1, wherein the compound of Formula A is treated with the compound of Formula B or a salt thereof in the presence of a first base.

15.-17. (canceled)

18. The method of claim 1, wherein the compound of Formula B is a salt, comprising a coformer.

19.-21. (canceled)

22. The method of claim 1, wherein the treatment of the compound of Formula A with the compound of Formula B or a salt thereof provides a compound of Formula C-A: 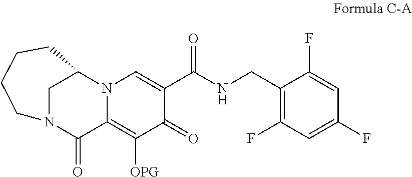
wherein deprotection of the compound of Formula C-A provides the compound of Formula I-A: 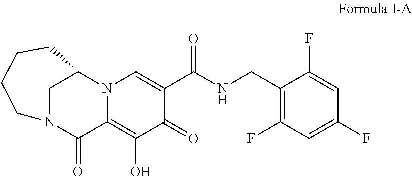 [SMILES: O=C(NCc1c(F)cc(F)cc1F)c1cn2c(c(O)c1=O)C(=O)N1CCCC[C@H]2C1]
or a salt thereof.

23.-46. (canceled)

47. The method of claim 1, further comprising preparing the compound of Formula A by reacting a compound of Formula D: 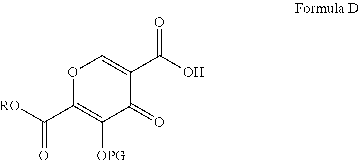
with a compound of Formula E: 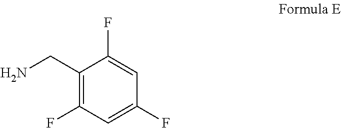 [SMILES: NCc1c(F)cc(F)cc1F]
or a salt thereof in the presence of a first coupling agent.

48.-50. (canceled)

51. The method of claim 47, wherein the compound of Formula D and the compound of Formula E or a salt thereof are reacted further in the presence of a first catalyst.

52.-54. (canceled)

55. The method of claim 47, wherein the compound of Formula D and the compound of Formula E or a salt thereof are reacted further in the presence of a second base.

56.-70. (canceled)

71. The method of claim 1, further comprising preparing the compound of Formula A by treating a compound of Formula F: 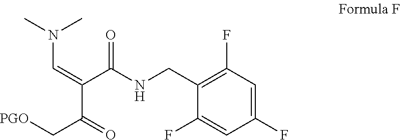
with a third base and a dicarbonyl reagent.

72.-77. (canceled)

78. The method of claim 71, wherein the compound of Formula F is further treated with a first promoter.

79.-88. (canceled)

89. The method of claim 71, further comprising preparing the compound of Formula F by treating a compound of Formula G: 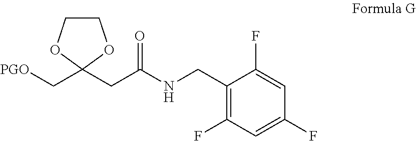
with a first acid.

90.-102. (canceled)

103. The method of claim 89, wherein treatment of the compound of Formula G with the first acid provides a compound of Formula H: 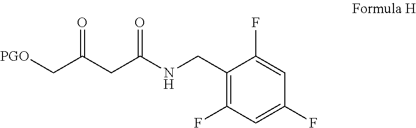
and wherein the compound of Formula H is treated with an electrophile and a second acid to provide the compound of Formula F.

104.-109. (canceled)

110. The method of claim 103, wherein the compound of Formula H is treated with the electrophile and the second acid further in the presence of a fourth base.

111.-120. (canceled)

121. The method of claim 89, further comprising preparing the compound of Formula G by treating a compound of Formula J: 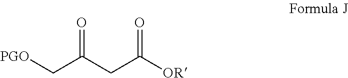
with a compound of Formula K: 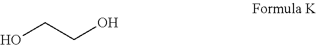
in the presence of a third acid and a second promoter;
wherein R′ is C₁₋₆ alkyl, C₆₋₁₀ aryl-C₁₋₄ alkylene, or C₆₋₁₀ aryl.

122.-133. (canceled)

134. The method of claim 121, wherein treatment of the compound of Formula J with the compound of Formula K provides a compound of Formula L: 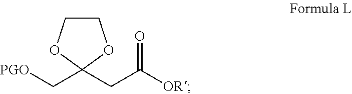
and wherein the method further comprises treating the compound of Formula L with a fifth base.

135.-141. (canceled)

142. The method of claim 134, wherein the compound of Formula L is treated with the fifth base further in the presence of a second catalyst.

143.-148. (canceled)

149. The method of claim 134, wherein treatment of the compound of Formula L with the fifth base provides a compound of Formula M: 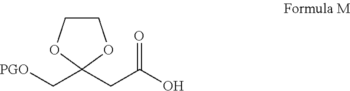
and wherein the method further comprises treating the compound of Formula M with a compound of Formula E: 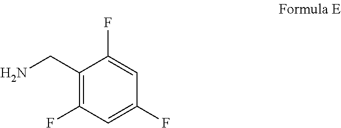 [SMILES: NCc1c(F)cc(F)cc1F]
or a salt thereof in the presence of a second coupling agent.

150.-159. (canceled)

160. The method of claim 1, wherein PG is benzyl or methyl.

161.-162. (canceled)

163. The method of claim 1, wherein R is methyl, ethyl, benzyl, or phenyl.

164. (canceled)

165. The method of claim 121, wherein R′ is methyl, ethyl, isopropyl, tert-butyl, benzyl, or phenyl.

166. (canceled)

167. The method of claim 1, wherein the compound of Formula B is a compound of Formula B-A: 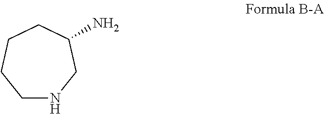
or a salt thereof.
